SEQUENCE LISTING (41 sequences)

SEQ ID NO: 1 - DNA, 7535 bp, Homo sapiens
  misc_feature: Homo sapiens chromosome 19, GRCh38.p7 Primary Assembly apoE3 (DNA) Accession number FJ525876.1
cagcggatccttgatgctgccttaagtccggctcagaggggcgcagcgtggcctggggtcgctatcttcccatccggaacatctgccctgctgggggacactacgggccttcccttgcctgagggtagggtctcaaggtcacttgcccccagcttgacctggccggagtggctatagaggactttgtccctgcagactgcagcagcagagatgacactgtctctgagtgcagagatgggggcagggagctgggagagggttcaagctactggaacagcttcagaacaactagggtactaggaactgctgtgtcagggagaaggggctcaaggactcgcaggcctgggaggaggggcctaggccagccatgggagttgggtcacctgtgtctgaggacttggtgctgtctggattttgccaacctagggctggggtcagctgatgcccaccacgactcccgagcctccaggaactgaaaccctgtctgcccccagggtctggggaaggaggctgctgagtagaaccaaccccaggttaccaaccccacctcagccaccccttgccagccaaagcaaacaggcccggcccggcactgggggttccttctcgaaccaggagttcagcctcccctgacccgcagaatcttctgatcccacccgctccaggagccaggaatgagtcccagtctctcccagttctcactgtgtggttttgccattcgtcttgctgctgaaccacgggtttctcctctgaaacatctgggatttataacagggcttaggaaagtgacagcgtctgagcgttcactgtggcctgtccattgctagccctaacataggaccgctgtgtgccagggctgtcctccatgctcaatacacgttagcttgtcaccaaacatacccgtgccgctgctttcccagtctgatgagcaaaggaacttgatgctcagagaggacaagtcatttgcccaaggtcacacagctggcaactggcagagccaggattcacgccctggcaatttgactccagaatcctaaccttaacccagaagcacggcttcaagcccctggaaaccacaatacctgtggcagccagggggaggtgctggaatctcatttcacatgtggggagggggctcccctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtctcaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtctggattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgatttcctctaagccccagcctcagtttctctttctgcccacatactggccacacaattctcagccccctcctctccatctgtgtctgtgtgtatctttctctctgcccttttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttgggggggggggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctgggctcaagagatcctccgccatcggcctcccaaagtgctgggattccaggcatggggctccgagcccggcctgcccaacttaataatacttgttcctcagagttgcaactccaaatgacctgagattggtgcctttattctaagctattttcattttttttctgctgtcattattctcccccttctctcctccagtcttatctgatatctgcctccttcccacccaccctgcaccccatcccacccctctgtctctccctgttctcctcaggagactctggcttcctgttttcctccacttctatcttttatctctccctcctacggtttcttttctttctccccggcctgcttgtttctcccccaacccccttcatctggatttcttcttctgccattcagtttggtttgagctctctgcttctccggttccctctgagctagctgtcccttcacccactgtgaactgggtttccctgcccaaccctcattctctttctttctttctttttttttttttttttttttttttttttttgagacagagtcttgctctgttgcccagcctggagtgcagtggtgcaatcttggttcactgcaacctccacttcccagattcaagcaattctcctgcctcagcctccagagtagctgggattacaggcgtgtcccaccacacccgactaatttttgtatttttggtagagacaaggcttcggcattgttggccaggcaggtctcgaactcctgacctcaagtaatctgcctgcctcaccctcccaaagtgctgggattacaggcatgagccacctcacccggaccatccctcattctccatcctttcctccagttgtgatgtctacccctcatgtttcccaacaagcctactgggtgctgaatccaggctgggaagagaagggagcggctcttctgtcggagtctgcaccaggcccatgctgagacgagagctggcgctcagagaggggaagcttggatggaagcccaggagccgccggcactctcttcccctcccaccccctcagttctcagagacggggaggagggttcccaccaacgggggacaggctgagacttgagcttgtatctcctgggccagctgcaacatctgcttgtccctctgcccatcttggctcctgcacaccctgaacttggtgctt

SEQ ID NO: 2 - protein, 317 aa, Homo sapiens [sequence omitted]

SEQ ID NO: 3 - DNA, 5515 bp, Homo sapiens
  misc_feature: ApoE4 (DNA) Genbank M10065.1
ggaacttgatgctcagagaggacaagtcatttgcccaaggtcacacagctggcaactggcagacgagattcacgccctggcaatttgactccagaatcctaaccttaacccagaagcacggcttcaagccctggaaaccacaatacctgtggcagccagggggaggtgctggaatctcatttcacatgtggggagggggctcctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtctcaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtctgtattactgggcgaggtgtcctcccttcctggggactgtggggggtggtcaaaagacctctatgccccacctccttcctccctctgccctgctgtgcctggggcagggggagaacagcccacctcgtgactgggctgcccagcccgccctatccctgggggagggggcgggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaagccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcattatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttgggggcgctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcagacagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggccggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtagctagatgcctggacggggtcagaaggaccctgacccgaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagcaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctcccccgcctccccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgatttcctctaagccccagcctcagtttctctttctgcccacatactgccacacaattctcagccccctcctctccatctgtgtctgtgtgtatctttctctctgccctttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttgggggggggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctggctcaagagatcctccgccatcggcctcccaaagtgctgggattccaggcatgggctccgagcggcctgcccaacttaataatattgttcctagagttgcactc

SEQ ID NO: 4 - protein, 317 aa, Homo sapiens [sequence omitted]

SEQ ID NO: 5 - DNA, 4215 bp, Artificial Sequence
  VRER Cas9 (DNA)
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcaggatga

SEQ ID NO: 6 - protein, 1404 aa, Artificial Sequence
  VRER Cas9 (Protein)
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGDGSPKKKRKVSSDYKDHDGDYKDHDIDYKDDDDKAAG

SEQ ID NO: 7 - DNA, 7614 bp, Artificial Sequence
  MSP1101 plasmid (DNA)
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcaggatgaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatc

SEQ ID NO: 8 - DNA, 20 bp, Artificial Sequence
  gRNA for APOE4
  misc_feature: gRNA for APOE4
gggcgcggacatggaggacg

SEQ ID NO: 9 - DNA, 24 bp, Artificial Sequence
  gRNA oligo 1
caccgggcgcggacatggaggacg

SEQ ID NO: 10 - DNA, 24 bp, Artificial Sequence
  gRNA oligo 2
cccgcgcctgtacctcctgccaaa

SEQ ID NO: 11 - DNA, 20 bp, Artificial Sequence
  Forward primer
ggacgagaccatgaaggagt

SEQ ID NO: 12 - DNA, 19 bp, Artificial Sequence
  Reverse primer
gcagcttgcgcaggtggga

SEQ ID NO: 13 - DNA, 64 bp, Artificial Sequence
  ApoE4 5′-3′ FRAGMENT OF FIGURE 2
gctgcaggcggcgcaggcccggctgggcgcggacatggaggacgtgcgcggccgcctggtgcag

SEQ ID NO: 14 - DNA, 64 bp, Artificial Sequence
  ApoE4 3′-5′ FRAGMENT OF FIGURE 2
cgacgtccgccgcgtccgggccgacccgcgcctgtacctcctgcacacgccggcggaccacgtc

SEQ ID NO: 15 - DNA, 64 bp, Artificial Sequence
  ApoE3 5′-3′ FRAGMENT OF FIGURE 2
gctgcaggcggcgcaggcccggctgggcgcggacatggaggacgtgtgcggccgcctggtgcag

SEQ ID NO: 16 - DNA, 64 bp, Artificial Sequence; identical to SEQ ID NO: 14
  ApoE3 3′-5′ FRAGMENT OF FIGURE 2

SEQ ID NO: 17 - DNA, 242 bp, Artificial Sequence
  ApoE4 PCR OF FIGURE 3
ggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgc

SEQ ID NO: 18 - DNA, 19 bp, Artificial Sequence
  ApoE4 proto-spacer sequence
ggcgcggacatggaggacg

SEQ ID NO: 19 - DNA, 19 bp, Artificial Sequence
  19bp gRNA
gggcgcggacatggaggac

SEQ ID NO: 20 - DNA, 18 bp, Artificial Sequence
  18bp gRNA
gggcgcggacatggagga

SEQ ID NO: 21 - DNA, 17 bp, Artificial Sequence
  17bp gRNA
gggcgcggacatggagg

SEQ ID NO: 22 - DNA, 22 bp, Artificial Sequence
  22bp gRNA
ctgggcgcggacatggaggacg

SEQ ID NO: 23 - DNA, 20 bp, Artificial Sequence
  gRNA-1
gacagccgtgcccgcgtctc

SEQ ID NO: 24 - DNA, 20 bp, Artificial Sequence
  gRNA-2
cgcaggtgggaggcgaggcg

SEQ ID NO: 25 - DNA, 197 bp, Artificial Sequence
  ApoE4 allele fragment
gaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcg

SEQ ID NO: 26 - DNA, 144 bp, Artificial Sequence
  donor sequence
gaggagacgcgggcacggctgtccaaggagctgcaggcggcgcaggcccggctgggcgcggacatggaggacgtctgcgggcggctggtgcagtaccgcggcgaggtgcaggccatgctcggccagagcaccgaggagctgcgg

SEQ ID NO: 27 - DNA, 14873 bp, Artificial Sequence
  VRER lentiCRISPR v2
tgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacggttgtaaatgagcacacaaaatacacatgctaaaatattatattctatgacctttataaaatcaaccaaaatcttctttttaataactttagtatcaataattagaatttttatgttcctttttgcaaacttttaataaaaatgagcaaaataaaaaaacgctagttttagtaactcgcgttgttttcttcacctttaataatagctactccaccacttgttcctaagcggtcagctcctgcttcaatcattttttgagcatcttcaaatgttctaactccaccagctgctttaactaaagcattgtctttaacaactgacttcattagtttaacatcttcaaatgttgcacctgattttgaaaatcctgttgatgttttaacaaattctaatccagcttcaacagctatttcacaagctttcatgatttcttcttttgttaataaacaattttccataatacatttaacaacatgtgatccagctgctttttttacagctttcatgtcttctaaaactaattcataatttttgtcttttaatgcaccaatatttaataccatatcaatttctgttgcaccatctttaattgcttcagaaacttcgaatgcttttgtagctgttgtgcatgcacctagaggaaaacctacaacatttgttattcctacatttgtgccttttaataattctttacaatagcttgttcaatatgaattaacacaaactgttgcaaaatcaaattcaattgcttcatcacataattgtttaatttcagctttcgtagcatcttgttttaataatgtgtgatctatatatttgtttagtttcattttttctcctatatattcatttttaattttaattctttaataatttcgtctactttaactttagcgttttgaacagattcaccaacacctataaaataaatttttagtttaggttcagttccacttgggcgaacagcaaatcatgacttatcttctaaataaaattttagtaagtcttgtcctggcatattatacattccatcgatgtagtcttcaacattaacaactttaagtccagcaatttgagttaagggtgttgctctcaatgatttcattaatggttcaatttttaatttcttttcttctggtttaaaattcaagtttaaagtgaaagtgtaatatgcacccatttctttaaataaatcttctaaatagtctactaatgttttattttgttttttataaaatcaagcagcctctgctattaatatagaagcttgtattccatctttatctctagctgagtcatcaattacatatccataactttcttcataagcaaaaacaaaatttaatccgttatcttcttctttagcaatttctctacccattcatttaaatccagttaaagtttttacaatattaactccatatttttcatgagcgattctatcacccaaatcacttgttacaaaacttgaatatagagccggattttttggaatgctatttaagcgttttagatttgataattttcaatcaattaaaattggtcctgtttgatttccatctaatcttacaaaatgaccatcatgttttattgccattccaaatctgtcagcatctgggtcattcataataataatatctgcatcatgtttaataccatattcaagcggtatttttcatgcaggatcaaattctggatttggatttacaacatttttaaatgtttcatcttcaaatgcatgctcttcaacctcaataacgttatatcctgattcacgtaatatttttggggtaaatttagttcctgttccattaactgcgctaaaaataatttttaaatcttttttagcttcttgctcttttttgtacgtctctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgucagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgcccgcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaaggagtacaggagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 28 - protein, 1368 aa, Streptococcus pyogenes [sequence omitted]

SEQ ID NO: 29 - DNA, 4104 bp, Streptococcus pyogenes
  misc_feature: WT spCAS9
atggataaaaaatacagcattggtctggacattggcacgaatagcgttggttgggcagtgattaccgatgaatacaaagtcccgtcgaaaaaattcaaagtgctgggtaacaccgatcgccatagcattaagaaaaacctgatcggtgcgctgctgtttgattctggcgaaaccgcggaagcaacgcgtctgaaacgtaccgcacgtcgccgttacacgcgccgtaaaaatcgtatttgctatctgcaggaaatctttagcaacgaaatggcgaaagtcgatgactcatttttccaccgcctggaagaatcgtttctggtggaagaagataaaaaacatgaacgtcacccgattttcggcaatatcgttgatgaagtcgcgtaccatgaaaaatatccgacgatttaccacctgcgtaaaaaactggtggattctaccgacaaagccgatctgcgcctgatttatctggcactggctcatatgatcaaatttcgtggtcacttcctgattgaaggcgacctgaacccggataatagtgacgtcgataaactgtttattcagctggtgcaaacctataatcagctgttcgaagaaaacccgatcaatgcaagtggtgttgatgcgaaagccattctgtccgctcgcctgagtaaatcccgccgtctggaaaacctgattgcacagctgccgggtgaaaagaaaaacggtctgtttggcaatctgatcgctctgtcactgggcctgacgccgaactttaaatcgaatttcgacctggcagaagatgctaaactgcagctgagcaaagatacctacgatgacgatctggacaacctgctggcgcaaattggcgaccagtatgccgacctgtttctggcggccaaaaatctgtcagatgccattctgctgtcggacatcctgcgcgtgaacaccgaaatcacgaaagcgccgctgtcagcctcgatgattaaacgctacgatgaacatcaccaggacctgaccctgctgaaagcactggttcgtcagcaactgccggaaaaatacaaagaaattttctttgaccaaagtaaaaatggttatgcaggctacatcgatggcggtgcttcccaggaagaattctacaaattcatcaaaccgatcctggaaaaaatggatggtacggaagaactgctggtgaaactgaatcgtgaagatctgctgcgtaaacaacgcacctttgacaacggtagcattccgcatcagatccacctgggcgaactgcatgcgattctgcgccgtcaggaagatttttatccgttcctgaaagacaaccgtgaaaaaatcgaaaaaatcctgacgtttcgcatcccgtattacgttggtccgctggcacgtggtaatagccgcttcgcatggatgacccgcaaatctgaagaaaccattacgccgtggaactttgaagaagtggttgataaaggcgcaagcgctcagtcttttatcgaacgtatgaccaatttcgataaaaacctgccgaatgaaaaagtgctgccgaaacattctctgctgtatgaatactttaccgtttacaacgaactgacgaaagtgaaatatgttaccgagggtatgcgcaaaccggcgtttctgagtggcgaacagaaaaaagccattgtggatctgctgttcaaaaccaatcgtaaagttacggtcaaacagctgaaagaagattacttcaagaaaattgaatgtttcgacagcgtggaaatttctggtgttgaagatcgtttcaacgcctctctgggcacctatcatgacctgctgaaaatcatcaaagacaaagattttctggataacgaagaaaacgaagacattctggaagatatcgtgctgaccctgacgctgttcgaagatcgtgaaatgattgaagaacgcctgaaaacgtacgcacacctgtttgacgataaagttatgaaacagctgaaacgccgtcgctataccggttggggccgtctgagccgcaaactgattaatggtatccgcgataaacaatcaggcaaaacgattctggatttcctgaaatcggacggctttgccaaccgtaatttcatgcagctgatccatgacgattccctgacctttaaagaagacattcagaaagcacaagtgtcaggtcaaggcgattcgctgcatgaacacattgcgaacctggccggttcaccggctatcaaaaaaggcatcctgcagaccgtgaaagtcgtggatgaactggtgaaagttatgggtcgtcacaaaccggaaaacattgttatcgaaatggcgcgcgaaaatcagaccacgcaaaaaggccagaaaaactcgcgtgaacgcatgaaacgcattgaagaaggtatcaaagaactgggcagccagattctgaaagaacatccggtcgaaaacacccagctgcaaaatgaaaaactgtacctgtattacctgcaaaatggtcgtgacatgtatgtggatcaggaactggacatcaaccgcctgtctgactatgatgtcgaccacattgtgccgcagagctttctgaaagacgattctatcgataacaaagttctgacccgtagtgataaaaaccgcggcaaaagcgacaatgtcccgtctgaagaagttgtgaagaaaatgaaaaactactggcgtcaactgctgaatgcgaaactgattacgcagcgtaaattcgataacctgaccaaagcggaacgcggcggtctgtccgaactggataaagccggttttatcaaacgtcaactggttgaaacccgccagattacgaaacatgtcgcccagatcctggattcacgcatgaacacgaaatacgacgaaaacgataaactgatccgtgaagtcaaagtgatcaccctgaaaagtaaactggtttccgatttccgtaaagactttcagttctacaaagtccgcgaaattaacaattaccatcacgcacacgatgcttatctgaatgcagtggttggtaccgctctgatcaaaaaatatccgaaactggaaagcgaatttgtgtatggcgattacaaagtctatgacgtgcgcaaaatgattgcgaaatccgaacaggaaatcggcaaagcgaccgccaaatactttttctattcaaacatcatgaactttttcaaaaccgaaattacgctggcaaatggtgaaattcgtaaacgcccgctgatcgaaaccaacggtgaaacgggcgaaattgtgtgggataaaggccgtgacttcgcgaccgttcgcaaagtcctgtcgatgccgcaagtgaatatcgtgaagaaaaccgaagtgcagacgggcggttttagtaaagaatccatcctgccgaaacgtaacagcgataaactgattgcgcgcaaaaaagattgggacccgaaaaaatacggcggttttgatagtccgacggttgcatattccgtcctggtcgtggctaaagtcgaaaaaggtaaaagtaaaaaactgaaatccgtgaaagaactgctgggcattaccatcatggaacgtagctcttttgagaaaaacccgattgacttcctggaagccaaaggttacaaagaagtgaaaaaagatctgatcatcaaactgccgaaatatagcctgttcgaactggaaaacggccgtaaacgcatgctggcatctgctggtgaactgcagaaaggcaatgaactggcactgccgagtaaatatgttaactttctgtacctggctagccattatgaaaaactgaaaggttctccggaagataacgaacagaaacaactgttcgtcgaacaacataaacactacctggatgaaatcatcgaacagatctcagaattctcgaaacgcgtgattctggcggatgccaatctggacaaagttctgagcgcgtataacaaacatcgtgataaaccgattcgcgaacaggccgaaaatattatccacctgtttaccctgacgaacctgggcgcaccggcagcttttaaatacttcgataccacgatcgaccgtaaacgctatacctcaacgaaagaagttctggatgctaccctgattcatcaatcgatcaccggtctgtatgaaacgcgtattgatctgagtcagctgggcggtgac

SEQ ID NO: 30 - DNA, 19 bp, Artificial Sequence
  3-5 SEQUENCE COMPLEMENTARY TO THE SEQUENCE RECOGNIZED BY CAS9
ccgcgcctgtacctcctgc

SEQ ID NO: 31 - DNA, 21 bp, Artificial Sequence
  gRNA for the ApoE rs28931579 SNP 5′ to 3′
gcttcggcgttcagtgattgt

SEQ ID NO: 32 - DNA, 49 bp, Artificial Sequence
  ApoE rs28931579 5′-3′ FRAGMENT OF FIGURE 9
ggcaccagcgccgcccctgtgccccgcgacaatcactgaacgccgaagc

SEQ ID NO: 33 - DNA, 49 bp, Artificial Sequence
  ApoE rs28931579 3′-5′ FRAGMENT OF FIGURE 9
ccgtggtcgcggcggggacacggggcgctgttagtgacttgcggcttcg

SEQ ID NO: 34 - DNA, 49 bp, Artificial Sequence
  ApoE WT 5′-3′ FRAGMENT OF FIGURE 9
ggcaccagcgccgcccctgtgcccagcgacaatcactgaacgccgaagc

SEQ ID NO: 35 - DNA, 49 bp, Artificial Sequence
  ApoE WT 3′-5′ FRAGMENT OF FIGURE 9
ccgtggtcgcggcggggacacgggtcgctgttagtgacttgcggcttcg

SEQ ID NO: 36 - DNA, 20 bp, Artificial Sequence
  protospacer
cttcggcgttcagtgattgt

SEQ ID NO: 37 - DNA, 20 bp, Artificial Sequence
  Complementary to protospacer
acaatcactgaacgccgaag

SEQ ID NO: 38 - DNA, 7535 bp, Homo sapiens
  misc_feature: ApoE2
cagcggatccttgatgctgccttaagtccggctcagaggggcgcagcgtggcctggggtcgctatcttcccatccggaacatctgccctgctgggggacactacgggccttcccttgcctgagggtagggtctcaaggtcacttgcccccagcttgacctggccggagtggctatagaggactttgtccctgcagactgcagcagcagagatgacactgtctctgagtgcagagatgggggcagggagctgggagagggttcaagctactggaacagcttcagaacaactagggtactaggaactgctgtgtcagggagaaggggctcaaggactcgcaggcctgggaggaggggcctaggccagccatgggagttgggtcacctgtgtctgaggacttggtgctgtctggattttgccaacctagggctggggtcagctgatgcccaccacgactcccgagcctccaggaactgaaaccctgtctgcccccagggtctggggaaggaggctgctgagtagaaccaaccccaggttaccaaccccacctcagccaccccttgccagccaaagcaaacaggcccggcccggcactgggggttccttctcgaaccaggagttcagcctcccctgacccgcagaatcttctgatcccacccgctccaggagccaggaatgagtcccagtctctcccagttctcactgtgtggttttgccattcgtcttgctgctgaaccacgggtttctcctctgaaacatctgggatttataacagggcttaggaaagtgacagcgtctgagcgttcactgtggcctgtccattgctagccctaacataggaccgctgtgtgccagggctgtcctccatgctcaatacacgttagcttgtcaccaaacatacccgtgccgctgctttcccagtctgatgagcaaaggaacttgatgctcagagaggacaagtcatttgcccaaggtcacacagctggcaactggcagagccaggattcacgccctggcaatttgactccagaatcctaaccttaacccagaagcacggcttcaagcccctggaaaccacaatacctgtggcagccagggggaggtgctggaatctcatttcacatgtggggagggggctcccctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtctcaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtctggattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgatttcctctaagccccagcctcagtttctctttctgcccacatactggccacacaattctcagccccctcctctccatctgtgtctgtgtgtatctttctctctgcccttttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttgggggggggggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctgggctcaagagatcctccgccatcggcctcccaaagtgctgggattccaggcatggggctccgagcccggcctgcccaacttaataatacttgttcctcagagttgcaactccaaatgacctgagattggtgcctttattctaagctattttcattttttttctgctgtcattattctcccccttctctcctccagtcttatctgatatctgcctccttcccacccaccctgcaccccatcccacccctctgtctctccctgttctcctcaggagactctggcttcctgttttcctccacttctatcttttatctctccctcctacggtttcttttctttctccccggcctgcttgtttctcccccaacccccttcatctggatttcttcttctgccattcagtttggtttgagctctctgcttctccggttccctctgagctagctgtcccttcacccactgtgaactgggtttccctgcccaaccctcattctctttctttctttctttttttttttttttttttttttttttttttgagacagagtcttgctctgttgcccagcctggagtgcagtggtgcaatcttggttcactgcaacctccacttcccagattcaagcaattctcctgcctcagcctccagagtagctgggattacaggcgtgtcccaccacacccgactaatttttgtatttttggtagagacaaggcttcggcattgttggccaggcaggtctcgaactcctgacctcaagtaatctgcctgcctcaccctcccaaagtgctgggattacaggcatgagccacctcacccggaccatccctcattctccatcctttcctccagttgtgatgtctacccctcatgtttcccaacaagcctactgggtgctgaatccaggctgggaagagaagggagcggctcttctgtcggagtctgcaccaggcccatgctgagacgagagctggcgctcagagaggggaagcttggatggaagcccaggagccgccggcactctcttcccctcccaccccctcagttctcagagacggggaggagggttcccaccaacgggggacaggctgagacttgagcttgtatctcctgggccagctgcaacatctgcttgtccctctgcccatcttggctcctgcacaccctgaacttggtgctt

SEQ ID NO: 39 - protein, 317 aa, Homo sapiens [sequence omitted]

SEQ ID NO: 40 - DNA, 7535 bp, Homo sapiens
  misc_feature: ApoE rs28931579 SNP
cagcggatccttgatgctgccttaagtccggctcagaggggcgcagcgtggcctggggtcgctatcttcccatccggaacatctgccctgctgggggacactacgggccttcccttgcctgagggtagggtctcaaggtcacttgcccccagcttgacctggccggagtggctatagaggactttgtccctgcagactgcagcagcagagatgacactgtctctgagtgcagagatgggggcagggagctgggagagggttcaagctactggaacagcttcagaacaactagggtactaggaactgctgtgtcagggagaaggggctcaaggactcgcaggcctgggaggaggggcctaggccagccatgggagttgggtcacctgtgtctgaggacttggtgctgtctggattttgccaacctagggctggggtcagctgatgcccaccacgactcccgagcctccaggaactgaaaccctgtctgcccccagggtctggggaaggaggctgctgagtagaaccaaccccaggttaccaaccccacctcagccaccccttgccagccaaagcaaacaggcccggcccggcactgggggttccttctcgaaccaggagttcagcctcccctgacccgcagaatcttctgatcccacccgctccaggagccaggaatgagtcccagtctctcccagttctcactgtgtggttttgccattcgtcttgctgctgaaccacgggtttctcctctgaaacatctgggatttataacagggcttaggaaagtgacagcgtctgagcgttcactgtggcctgtccattgctagccctaacataggaccgctgtgtgccagggctgtcctccatgctcaatacacgttagcttgtcaccaaacatacccgtgccgctgctttcccagtctgatgagcaaaggaacttgatgctcagagaggacaagtcatttgcccaaggtcacacagctggcaactggcagagccaggattcacgccctggcaatttgactccagaatcctaaccttaacccagaagcacggcttcaagcccctggaaaccacaatacctgtggcagccagggggaggtgctggaatctcatttcacatgtggggagggggctcccctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtctcaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtctggattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgccccgcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgatttcctctaagccccagcctcagtttctctttctgcccacatactggccacacaattctcagccccctcctctccatctgtgtctgtgtgtatctttctctctgcccttttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttgggggggggggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctgggctcaagagatcctccgccatcggcctcccaaagtgctgggattccaggcatggggctccgagcccggcctgcccaacttaataatacttgttcctcagagttgcaactccaaatgacctgagattggtgcctttattctaagctattttcattttttttctgctgtcattattctcccccttctctcctccagtcttatctgatatctgcctccttcccacccaccctgcaccccatcccacccctctgtctctccctgttctcctcaggagactctggcttcctgttttcctccacttctatcttttatctctccctcctacggtttcttttctttctccccggcctgcttgtttctcccccaacccccttcatctggatttcttcttctgccattcagtttggtttgagctctctgcttctccggttccctctgagctagctgtcccttcacccactgtgaactgggtttccctgcccaaccctcattctctttctttctttctttttttttttttttttttttttttttttttgagacagagtcttgctctgttgcccagcctggagtgcagtggtgcaatcttggttcactgcaacctccacttcccagattcaagcaattctcctgcctcagcctccagagtagctgggattacaggcgtgtcccaccacacccgactaatttttgtatttttggtagagacaaggcttcggcattgttggccaggcaggtctcgaactcctgacctcaagtaatctgcctgcctcaccctcccaaagtgctgggattacaggcatgagccacctcacccggaccatccctcattctccatcctttcctccagttgtgatgtctacccctcatgtttcccaacaagcctactgggtgctgaatccaggctgggaagagaagggagcggctcttctgtcggagtctgcaccaggcccatgctgagacgagagctggcgctcagagaggggaagcttggatggaagcccaggagccgccggcactctcttcccctcccaccccctcagttctcagagacggggaggagggttcccaccaacgggggacaggctgagacttgagcttgtatctcctgggccagctgcaacatctgcttgtccctctgcccatcttggctcctgcacaccctgaacttggtgctt

SEQ ID NO: 41 - protein, 317 aa, Homo sapiens
  MISC_FEATURE: ApoE rs28931579 SNP
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPRDNH

(19) United States
(12) Patent Application Publication
(10) Pub. No.: US 2020/0046853 A1
(43) Pub. Date: Feb. 13, 2020

(54) COMPOSITIONS AND METHODS SPECIFICALLY TARGETING THE APOLIPOPROTEIN E4 (APOE4) AND USES THEREOF IN APOE4 ASSOCIATED CONDITIONS

(71) Applicant: Ramof at Tel-Aviv University Ltd., Tel-Aviv (IL)

(72) Inventors: David OFFEN, Tel-Aviv (IL); Roy RABINOWITZ, Tel-Aviv (IL); Tall BEN-ZUR, Tel-Aviv (IL); Daniel MICHAELSON, Tel-Aviv (IL)

(21) Appl. No.: 16/498,993

(22) PCT Filed: Mar. 29, 2018
(86) PCT No.: PCT/IL2018/050381

§ 371 Date: Sep. 27, 2019

Related U.S. Application Data
(60) Provisional application No. 62/478,217, filed on Mar. 29, 2017.

(51) Int. Cl. A61K 48/00; C12N 15/11; C12N 9/22; A61K 31/7088; A61K 38/46; A61P 25/28; C12Q 1/6883; C12N 15/90

(52) U.S. Cl. CPC A61K 48/0066; C12N 15/11; C12N 9/22; A61K 31/7088; A61K 38/465; C12Q 2600/156; C12Q 1/6883; C12N 15/907; C12N 2310/20; C12N 2800/80; A61P 25/28

(57) ABSTRACT

The invention relates to improvement and therapy of neuronal conditions involving neurodegenerative, inflammatory and vascular conditions. More specifically, the invention provides compositions and methods for specific targeted elimination of apolipoprotein E4 (apoE4), and uses thereof in the treatment and prevention of ApoE4 associated conditions.

### FIELD OF THE INVENTION

The invention relates to cognitive conditions improvement and therapy. More specifically, the invention provides compositions and methods for specific targeted elimination of pathogenic forms of apolipoprotein, specifically, apolipoprotein E4 (apoE4), and uses thereof in the treatment and prevention of ApoE4 associated conditions.

### BACKGROUND ART

References considered to be relevant as background to the presently disclosed subject matter are listed below:
[1] Corder E H, et al., Science 261:921-923 (1993)
[2] Saunders A M, et al. Neurology 43:1467-1472 (1993)
[3] Roses A D, et al., Mol Cell. 61(6):895-902 (1996)
[4] Liu C C, et al., Nat Rev 9:106-18 (2013)
[5] Chapman J, et al., Neurology 57:1482-5 (2001)
[6] ZhouW, et al., J Neurotrauma 25:279-90 (2008)
[7] Reiman E M, et al., Ann Neurol. 44:288-91 (1998)
[8] Shaw P, et al., Lancet Neurol. 6:494-500 (2007)
[9] Dean D C et al., JAMA Neurol. 71:11-22 (2014)
[10] Dounda J A, Charpentier E., Science 346:1258996-1-9 (2014)
[11] Komor A C, et al., Nature 533:420-424 (2016)
[12] Hirano et al., Molecular Cell 61:886-894 (2016)
[13] Kleinstiver B P, et al., Nature. doi: 10.1038/nature14592. (2015)
[14] Liraz et al., Mol Neurodegener. doi: 10.1186/1750-1326-8-16 (2013)
[15] Shalem O, et al. Science, 343, 83-7. doi:10.1126/science.1247005 (2014)
[16] Courtney D G, et al., Gene Therapy 23, 108-112 (2016).

Acknowledgement of the above references herein is not to be inferred as meaning that these are in any way relevant to the patentability of the presently disclosed subject matter.

### BACKGROUND OF THE INVENTION

Brain pathology of Alzheimer's diseases (AD) and the genetics of autosomal dominant familial AD have been the “lamp posts” under which the AD field has been looking for therapeutic targets. Although this approach still remains valid, none of the compounds tested to date have produced clinically meaningful results. This calls for developing complementary therapeutic approaches and AD targets. The apolipoprotein E4 (apoE4), is known as the most prevalent genetic risk factor for sporadic AD. More than half of these patients express the apoE4 protein; in addition this protein is known to increase the risk of prevalence for Alzheimer's disease since it lowers the age of onset by as much as 10-20 years [1], [2], [3].

The apoE4 genotype combines synergistically with atherosclerosis and peripheral vascular diseases and in addition to AD, is a risk factor for vascular dementia and cerebro-amyloid angiogenesis and for cardiovascular diseases [4]. This suggests that the effects of apoE4 in AD may also be related at least in part to apoE4-driven vascular pathology. Interestingly, apoE4 is a risk factor for additional neurodegenerative diseases [5] and is also associated with poor recovery following traumatic brain injury (TBI) compared with the other APOE alleles [6]. Furthermore, the increased risk for AD following TBI is significantly higher in apoE4 carriers than in subjects who carry other APOE alleles. Because more than 20% of the general population carries the apoE4 allele, anti-apoE4 therapy is expected to also have an important effect on the treatment of TBI.

The effects of the APOE E4 allele can also be detected in healthy cases. Structural MRI experiments revealed accelerated age related decreases in cortical thickness and the hippocampal volume of healthy apoE4 carriers, which correlate with diminished cognitive performance in these cases [7]. Positron emission tomography and magnetic resonance imaging (MRI) imaging studies revealed lower levels of cerebral glucose metabolism and impairments in functional connectivity in healthy young adults and children that carry the APOE ε4 allele [8]. Furthermore, specific brain MRI changes have recently been observed in the brains of APOE ε4 infants [9]. Taken together, these findings demonstrates that the effects of the APOE ε4 allele and of its protein product apoE4 start decades before the onset of AD and may also be related to neurodevelopmental alterations.

Pathological effects of apoE4 may be counteracted, either at the protein level, using anti-apoE4 antibodies, or mimetic peptides, or preferentially at the gene level. The discovery of the CRISPR technique enables genome editing with high precision and efficiency [10]. This technique is particularly suitable for editing the apoE gene since the pathologic isoform apoE4 differs from the benign isoform apoE3 by only one nucleotide [3]. CRISPR may be applied either for the conversion of apoE4 to apoE3 or for silencing the apoE4 gene. Recently, an attempt was made to convert the isoform apoE4 to apoE3 in vitro? which was found to provide only very low yields and resulted in the conversion of apoE4 to a new isoform termed apoE3′ whose biological activity remains to be determined [11]. This attempt to convert apoE4 was not yet applied in vivo.

CRISPR targeted elimination has been suggested for other diseases. For example, targeted elimination of a SNP in the KRT12 gene that encodes keratin 12, heterozygous disease causing has been shown using the CRISPR/Cas9 system. As this particular SNP has been associated with heterozygous disease, targeted elimination thereof was suggested as a therapeutic approach [16].

Thus, there is need in the art for efficient and specific methods and compositions that specifically target ApoE SNPs, for example, the ApoE ε4 allele. One such approach is eliminating the pathogenic forms of ApoE, and specifically, the ApoE4 expression and thus preventing the associated pathologic conditions caused thereby.

These objects are successfully addressed by the present invention that provides a novel and highly effective gene editing related methodology for silencing the expression of apoE4 in vivo.

### SUMMARY OF THE INVENTION

In a first aspect, the invention relates to a method for targeted elimination of at least one pathogenic form of Apolipoprotein E in a cell. More specifically, the method comprising the step of contacting said cell with an effective amount of the following elements: (a) at least one polypeptide comprising at least one clustered regulatory interspaced short palindromic repeat (CRISPR) associated (cas) protein, or any nucleic acid encoding said polypeptide. It should be noted that said cas protein specifically recognizes the 5′-NGCG-3′ (proto-spacer adjacent motif) PAM. The second element is (b) at least one nucleic acid sequence comprising at least one guide RNA (gRNA) that targets a protospacer located upstream to said PAM within the at least one pathogenic form of Apolipoprotein E or any nucleic acid sequence encoding said gRNA; or with a kit or composition comprising (a) and (b).

In some particular embodiments, the pathogenic form of ApoE may be the Apolipoprotein E 4 (ApoE4) protein. Thus, in certain embodiments, the method of the invention may comprise the step of contacting a cell with at least one Cas protein that specifically recognizes the 5′-NGCG-3′ PAM, or any nucleic acid encoding said polypeptide. It should be noted that the cell is further contacted with at least one gRNA that targets a protospacer located upstream to this PAM within the ApoEε4 allele or any nucleic acid sequence encoding said gRNA.

In a further aspect, the invention provides a method for treating, preventing, ameliorating, inhibiting or delaying the onset of a pathologic disorder associated with at least one pathogenic form of the Apo E protein in a mammalian subject. More specifically, the method of the invention comprises the step of administering a therapeutically effective amount of: (a) at least one polypeptide comprising at least one Cas protein that specifically recognizes the 5′-NGCG-3′ PAM, or any nucleic acid encoding said polypeptide Cas; and (b) at least one nucleic acid sequence comprising at least one gRNA that targets a protospacer located upstream to said PAM within the at least one pathogenic form of the ApoE allele, or any nucleic acid sequence encoding said gRNA; or a construct, vehicle, kit or composition comprising (a) and (b). In more specific embodiments, the invention provides a method for treating, preventing, ameliorating, inhibiting or delaying the onset of an ApoE4 associated pathologic condition or disease in a mammalian subject. More specifically, the method of the invention may comprise the step of administering a therapeutically effective amount of: (a) at least one polypeptide comprising at least one cas protein that recognizes the 5′-NGCG-3′ PAM, or any nucleic acid encoding said polypeptide; and (b) at least one nucleic acid sequence comprising at least one gRNA that targets a protospacer located upstream to said PAM within the ApoEε4 allele, or any nucleic acid sequence encoding said gRNA; or a kit, construct, vehicle or composition comprising (a) and (b).

In yet a further aspect, the invention relates to a pharmaceutical composition comprising a therapeutic effective amount of: (a) at least one polypeptide comprising at least one Cas protein that specifically recognizes the 5′-NGCG-3′ PAM, or any nucleic acid encoding said polypeptide Cas; and (b) at least one nucleic acid sequence comprising at least one gRNA that targets a protospacer located upstream to the PAM within at least one pathogenic allele of ApoE, or any nucleic acid sequence encoding said gRNA. In some alternative embodiments, the composition of the invention may comprise a kit comprising (a) and (b) or alternatively, a construct or vehicle that comprises nucleic acid sequences encoding (a) and (b). In yet some further embodiments, the composition of the invention may optionally further comprise at least one of pharmaceutically acceptable carrier/s, diluent/s and/or excipient/s. Still further in some specific embodiments, the composition of the invention may comprises (a) at least one polypeptide comprising at least one Cas protein that recognizes the 5′-NGCG-3′ PAM, or any nucleic acid encoding said Cas; and (b) at least one nucleic acid sequence comprising at least one gRNA that targets a protospacer located upstream to said PAM within the ApoEε4 allele, or any nucleic acid sequence encoding said gRNA; or a kit, vehicle or composition comprising (a) and (b). In yet some further aspect thereof, the invention provides the use of a therapeutic effective amount of (a) at least one polypeptide comprising at least one Cas protein, or any nucleic acid encoding said polypeptide, more particularly, the cas protein specifically recognizes the 5′-NGCG-3′ PAM; and (b) at least one nucleic acid sequence comprising at least one gRNA that targets a protospacer located upstream to said PAM within at least one pathogenic allele of ApoE, or any nucleic acid sequence encoding said gRNA, in the preparation of a composition for treating, preventing, ameliorating, inhibiting or delaying the onset of a pathologic condition or disease associated with a pathogenic form of the Apo E protein in a mammalian subject. In some specific embodiments, the invention provides the use of a therapeutic effective amount of (a) at least one polypeptide comprising at least one Cas protein that recognizes the 5′-NGCG-3′ PAM, or any nucleic acid encoding said polypeptide; and (b) at least one nucleic acid sequence comprising at least one gRNA that targets a protospacer located upstream to said PAM within the ApoEε4 allele, or any nucleic acid sequence encoding said gRNA; or a kit, vehicle or composition comprising (a) and (b) in the preparation of a composition for treating, preventing, ameliorating, inhibiting or delaying the onset of an ApoE4 associated pathologic condition or disease in a mammalian subject. It should be noted that the invention further provides (a) at least one polypeptide comprising at least one Cas protein, or any nucleic acid encoding said polypeptide, more particularly, the Cas protein specifically recognizes the 5′-NGCG-3′ PAM; and (b) at least one nucleic acid sequence comprising at least one gRNA that targets a protospacer located upstream to said PAM within at least one pathogenic allele of ApoE, or any nucleic acid sequence encoding said gRNA, for use in targeted elimination of at least one pathogenic allele of ApoE, and in further embodiments, for use in the treatment of disorders associated in at least one pathogenic allele of ApoE.

A further aspect of the invention provides a diagnostic method for detecting the presence of at least one pathogenic ApoE allele in a subject. More specifically, the method of the invention may comprise the following steps: In a first step (a), contacting at least one biological sample of the subject with an effective amount of: (i) at least one polypeptide comprising at least one nuclease-dead CRISPR-associated protein (dCas), or any nucleic acid encoding said polypeptide. It should be noted that the dCas protein specifically recognizes the 5′-NGCG-3′ PAM. The sample is further contacted with (ii) at least one gRNA that targets a protospacer located upstream to said PAM within the ApoE pathogenic allele, or any nucleic acid sequence encoding said gRNA.

In some embodiments the at least one of the dCas of (i) and the gRNA of (ii) may be either directly or indirectly attached, connected, conjugated, associated or fused to at least one detectable moiety. It should be understood that such step of contacting the dCas and gRNA with the biologic sample is performed to allow the formation of a dCas9/sgRNA complex in the sample. The second step (b), involves determining if at least one detectable signal from the at least one detectable moiety is detected in the sample of (a). In some embodiments, the detection of such detectable signal indicates the presence of at least one pathogenic ApoE allele in the sample, specifically a biological sample that comprise genomic DNA, and thereby, in the tested subject.

In yet a further aspect, the invention provides a diagnostic kit comprising: (a) at least one polypeptide comprising at least one dCas or any fusion protein thereof, or any nucleic acid sequence encoding the dCas polypeptide. It should be noted that the dCas protein specifically recognizes the 5′-NGCG-3′ PAM. The kit of the invention further comprises (b), at least one gRNA that targets a protospacer located upstream to the PAM within the ApoE pathogenic allele, or any nucleic acid sequence encoding this gRNA.

In some embodiments, at least one of the dCas of (a) and/or the gRNA of (b) may be either directly or indirectly attached, connected, conjugated, associated or fused to at least one detectable moiety.

These and other aspects of the invention will become apparent by the hand of the following drawings.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1D. Illustration of the locations of the platform and start point of each trail for the Morris Water Maze

The figure illustrates the location of the platform in days 1-4 (FIG. 1A), the probe test of day 5 (FIG. 1B), day 5 (FIG. 1C) and day 6 (FIG. 1D). Abbreviations: platform (P).

FIG. 2. Specific PAM distinguish between ApoE4 and ApoE3 isoforms

Figure shows comparison of the nucleic acid sequence of the ApoEε4 allele, as denoted by SEQ ID NO. 13 (5′ to 3′) and 14 (3′ to 5′), with the ApoEε3 allele as denoted by SEQ ID NO. 15 (5′ to 3′) and 16 (3′ to 5′), indicating the PAM specific sequence appears only at ApoE4 genotype. The gRNA designed by the invention is also indicated (as denoted by SEQ ID NO. 8).

FIG. 3. Schematic presentation of specificity verification

Schematic representation of the PCR product employed for DNA analysis of the apoE4 locus (comprising the nucleic acid sequence as denoted by SEQ ID NO. 17). Sequences bound by the forward and reverse primers (denoted by SEQ ID NO. 11 and 12, respectively), appear in bold and underlined. The site of the DSB (double strand break) is marked | and the restriction site of the BmgBI restriction enzyme, GACGTG is boxed. The PAM sequence TGCG appearing only in apoE4 is bold.

FIG. 4. Verifying the specific targeting of ApoE4

BmgBI restriction pattern reveals CRISPR activity on ApoE4 gene.

Lane 1, Gene direx 100 bp DNA ladder. Lane 2, ApoE3 control (without CRISPR treatment) uncut DNA a full length 242 bp amplicon is visible while in Lane 3 ApoE3 control cut DNA by BmgBI two smaller segments are appearing. Lanes 4 and 5, ApoE3 following treatment with CRISPR, uncut and cut DNA looks similar to lanes 2 and 3 (control, without CRISPR treatment). Lane 6, ApoE4 control uncut DNA, a full length 242 bp amplicon is visible. Lane 7, ApoE4 control, cut by BmgBI into two smaller segments. Lane 8, ApoE4 CRISPR uncut, DNA appears similar to the correlate uncut control group. Lane 9 ApoE4 CRISPR cut by BmgBI is reduced in comparison to the cutting of the correlate control group, indicating the deformation of the BmgBI recognition site by CRISPR-Cas9.

FIG. 5A-5B. Specific elimination of ApoE4 expression

Detected secreted ApoE3 and ApoE4 levels in cells medium using plasmid constructs.

FIG. 5A. shows gels of western blot analysis for detection of the ApoE3 and ApoE4 proteins. ApoE3 (first and second lanes) and ApoE4 (third and fourth lanes) with or without CRISPR treatment.

FIG. 5B. shows numerical analysis of western blot detection of ApoE3 and ApoE4 proteins.

FIG. 6A-6D. In-vitro lentivirus transfection of the CRISPR/Cas9 system provides a higher rate of ApoE4 depletion in comparison with plasmid transfection

FIG. 6A. shows the levels of ApoE4 protein in culture medium of untreated cells (control), cells treated by viral infection with the VRER SpCas9 with or without sgRNA by numerical analysis of western blot detection.

FIG. 6B. shows the levels of ApoE3 protein in culture medium of untreated cells (control), cells treated by viral infection with the VRER SpCas9 with or without sgRNA by numerical analysis of western blot detection.

FIG. 6C. shows a picture of the bands reflecting ApoE4 levels obtained by western blot in control cells versus cells treated by viral infection with CRISPR-Cas9 system.

FIG. 6D. shows a picture of the bands reflecting ApoE3 levels obtained by western blot in control cells versus cells treated by viral infection with CRISPR-Cas9 system. Abbreviations: reduction (reduc.).

FIG. 7A-7B. Mice treated with lentivirus preparation containing the CRISPR-Cas9 system showed improved performance at the Morris Water Maze test

FIG. 7A. is a graph representing the results of the Morris water maze test in ApoE4/E4 mice treated with CRISPR-Cas9 system (pink) showing improved performance in comparison with untreated mice (green-control) especially in the probe test in which mice were given one trial on the 5^{th} day to reach the location in which the platform used to be during days 1-4.

FIG. 7B. is a graph representing the results of the Morris water maze test in ApoE3/E3 mice treated with CRISPR-Cas9 system (blue) in comparison with untreated mice (red-control). No significant change is observed between the treated mice and the control mice during days 1-4 and the probe test.

FIG. 8A-8B. Cpf-1 based CRISPR design to convert ApoE4 to ApoE3

FIG. 8A. shows a fragment of the ApoE4 allele (as denoted by SEQ ID NO. 25). Cpf-1 PAM sequences (TTN) appears in the black boxes. gRNA1 (upstream to the SNP, bold and underlined) is 5′-GACAGCCGTGCCCGCGTCTC-3′ (as denoted by SEQ ID NO. 23) and gRNA2 (downstream to the SNP, bold and underlined) is 5′-CGCAGGTGGGAGGCGAGGCG-3′ (as denoted by SEQ ID NO. 24). The SNP (Cytosine) is indicated by an arrow. The WT sequence contains NotI recognition site (GCGGCCGC, bold and italic). Cpf-1 cleavage occurs 18 bases downstream to PAM on the PAM strand and 23 bases downstream to PAM on the targeted strand (as indicated by the | marks). The staggered cleavage leaves overhangs which will be paired by the donor sequence.

FIG. 8B. represents the donor sequence (as denoted by SEQ ID NO. 26). The donor sequence contains overhang ends to pair with the sticky ends of the cleaved DNA. It is designed to replace the ApoE4 cytosine with a tyrosine as in ApoE3, (indicated by an arrow). In order to enable further analysis, the donor sequence includes 2 bases replacement to destroy the NotI recognition site (downstream to the SNP-marked, bold and italic), and one base replacement (underlined and bold) to create AatII recognition site (upstream to the SNP, in the black box).

FIG. 9. Specific PAM distinguish between APOE allele rs28931579 and WT isoforms

Figure shows comparison of the nucleic acid sequence of the APOE rs28931579 allele containing an Adenine to Cytosine (A to C) replacement mutation (indicated in bold), as denoted by SEQ ID NO. 32 (5′ to 3′) and SEQ ID NO. 33 (3′ to 5′), with the WT ApoE allele as denoted by SEQ ID NO. 34 (5′ to 3′) and SEQ ID NO. 35 (3′ to 5′), indicating the PAM specific sequence (boxed) appears only at APOE rs28931579 genotype. The gRNA designed by the invention is also indicated (bold underlined, as denoted by SEQ ID NO. 31).

Other aspects of the invention will become apparent as the description proceeds.

### DETAILED DESCRIPTION OF THE INVENTION

Apolipoprotein E (ApoE) is a major cholesterol carrier that supports lipid transport and injury repair in the brain. The APOE gene is a 3.6 Kb gene on chromosome 19. It encodes a 299-amino acid protein with functionally significant variations in codons 112 (Cys/Arg), specifically, rs429358 (TGC→CGC, Cys112Arg), and 158 (Cys/Arg), specifically, rs7412 [symbol] GC_→ [symbol] GC, Cys 158 Arg), leading to 3 common isoforms: ApoE2, Cys112/Cys158 (T-T, encoded by the ApoE ε2 allele), ApoE3, Cys112/Arg158 (C-T, encoded by the ApoE ε3 allele), and ApoE4, Arg 112/Arg 158 (C-C, encoded by the ApoE ε4 allele). It should be noted that the amino acid sequence of these different alleles of ApoE (or in other words, ApoE variants) are disclosed in SEQ ID NO. 1 and 2 (nucleic acid and amino acid sequences, respectively, of ApoE3), SEQ ID NO. 3 and 4 (nucleic acid and amino acid sequences, respectively, of ApoE4), SEQ ID NO. 38 and 39 (nucleic acid and amino acid sequences, respectively, of ApoE2). It should be further noted that in these sequences, specifically, the amino acid sequences of ApoE3, ApoE4 and ApoE2, as denoted y SEQ ID NO. 2, 4 and 39, respectively, amino acid residue 112 is located at position 130, and residue 158, is located at position 176. The different variants of ApoE discussed herein are further disclosed in Table 1, herein after.

The present invention is therefore involved in manipulation of polymorphism in genes that contribute to pathogenic phenotype. Specifically, polymorphism in the ApoE gene that is associated with pathogenic SNPs, such as the ApoE4 that is associated with neurodegenerative disorders such as Alzheimer's disease. The term “polymorphism” as herein defined refers to a location in the sequence of a gene which varies within a population. A polymorphism is comprised of different “alleles”. For example, T at the particular SNP rs7412 ( [symbol] GC→ [symbol] GC, Cys 158 Arg) indicates that there is a variation between C and T at the nucleic acid base located at position 44908822 in chromosome 19, and position 5426 in the ApoE sequences of SEQ ID NO. 1, 3 AND 38, in the ApoE gene, at the codon encoding residue 158. Because the genotype is comprised of two separate alleles, an individual may be either homozygous or heterozygous for a certain polymorphism (e.g. for the above example, an individual may be either CC, CT or TT). Thus a polymorphism may relate, inter alia, to a single nucleotide polymorphism, as illustrated in the example above. It should be understood that “A” refers to adenine, “T” refers to thymine, “C” relates to cytosine and “G” refers to guanine.

In other words, subjects that carry the ApoE allele of the specific SNPs disclosed herein is associated with genetic predisposition to pathologic conditions associated therewith, specifically, AD. The term “genetic predisposition” or the term “genetic susceptibility” as herein defined refers to a genetic-based increase in the risk of developing a disease or to a genetic-based tendency to suffer from a particular condition. Therefore, manipulating the specific SNPs, for example by gene editing techniques suggested herein, may be applicable for preventing and treating these associated disorders.

The term “single nucleotide polymorphism” (SNP) as herein defined, refers to a single base change in the DNA sequence. For a base position with sequence alternatives in genomic DNA to be considered as a SNP, the least frequent allele (the “minor allele” as herein described) should have a frequency of 1% or greater. The most frequent allele is referred to as the “major allele”. SNPs are usually bi-allelic, mainly due to the low frequency of single nucleotide substitutions in DNA. As known to a person skilled in the art, the term “SNP” usually refers to the least frequent allele (i.e. the minor allele), when present in the genome either on both chromosomes (then an individual is said to be homozygous for a certain polymorphism) or on a single chromosome (then an individual is said to be heterozygous for a certain polymorphism).

Known specific SNPs are assigned with unique identifiers, usually referred to by accession numbers with a prefix such as “SNP”, “refSNP” or “rs”, as known to one of skill in the art. Single nucleotide polymorphism database (dbSNP) of nucleotide sequence variation is available on the NCBI website. As indicated above, the invention specifically refers to two SNPs of the ApoE gene, specifically, the rs429358 (TGC→CGC, Cys112Arg) and rs7412 (CGC→\sqrt{T}GC, Arg 158Cys).

ApoE is strongly expressed in brain and liver and transports lipids, including cholesterol, through the cerebrospinal fluid (CSF) and plasma. In the brain, ApoE is synthesized by astrocytes and microglia but can also be produced by neurons following injury. Cholesterol, an essential component of cell membranes and myelin, is required for development, maintenance and repair of myelin sheaths, neuronal membranes and synaptic connections. ApoE plays a significant role in supplying cholesterol for these processes. However, the ApoE4 isoform has been associated with pathogenic processes. For example, several studies associated low myelin repair and impaired synapses in apoE4 carriers. There are also evidences that apoE4 peptides specifically impair neuronal synapses and cholinergic functions. Several findings highlight an amyloid-independent mechanism by which apoE4 can trigger vascular pathology and neurodegeneration. Likewise, a large number of cognitive studies shows deficits in learning and memory abilities in apoE4 carriers.

Therefore, one of the objectives of the invention may be targeted elimination of the ApoE4 protein.

Thus, in a first aspect, the invention relates to a method for targeted elimination of at least one pathogenic form of at least one Apolipoprotein E protein in a cell. More specifically, the method comprising the step of contacting said cell with an effective amount of the following elements: (a) at least one polypeptide comprising at least one clustered regulatory interspaced short palindromic repeat (CRISPR) associated (Cas) protein, or any nucleic acid encoding the Cas polypeptide. It should be noted that the Cas protein specifically recognizes the 5′-NGCG-3′ (proto-spacer adjacent motif) PAM. The second element is (b) at least one nucleic acid sequence comprising at least one guide RNA (gRNA) that targets a protospacer located upstream to said PAM within the at least one pathogenic ApoE allele, or any nucleic acid sequence encoding this gRNA; or with a vector, construct, vehicle, kit or composition comprising (a) and (b). It should be noted that when both elements are provided as nucleic acid sequences they may be provided either separately in two or more nucleic acid molecules or alternatively, together in a single nucleic acid molecule that comprises both sequences, specifically, construct or any vehicle comprising both, (a) and (b).

In more specific aspects thereof, the invention relates to a method for targeted elimination of the Apolipoprotein E 4 (ApoE4) protein in a cell. More specifically, the method comprising the step of contacting said cell with an effective amount of the following elements: (a) at least one polypeptide comprising at least one Cas protein, or any nucleic acid encoding said polypeptide. It should be noted that said Cas protein specifically recognizes the 5′-NGCG-3′ PAM. The second element is (b) at least one nucleic acid sequence comprising at least one gRNA that targets a protospacer located upstream to the PAM within the ApoEε4 allele, or any nucleic acid sequence encoding said gRNA; or with a construct or any vehicle, kit or composition comprising (a) and (b).

The Wild Type human ApoE gene is disclosed by NCBI Reference Sequence: NC_000019.10. It should be noted that in some specific embodiments, the ApoE4 referred to herein relates to the human ApoE4. In yet some specific embodiments, the ApoE4 may be encoded by a nucleic acid sequence comprising the sequence as denoted by SEQ ID NO. 3. In yet some further and non-limiting embodiments, the ApoE4 may comprise the amino acid sequence as denoted by SEQ ID NO. 4.

In some further embodiments, the pathogenic form of ApoE, may be derived from the rs28931579 SNP. More specifically, the method of the invention may in some embodiments be suitable for targeted elimination of the ApoE rs28931579 allele that contains an Adenine to Cytosine (A to C) replacement mutation that leads to substitution of Serine 296 to Arginine 296 (Ser296Arg). It should be appreciated that this specific substitution is shown in SEQ ID NO. 41 at position 314. This particular SNP is also referred to herein as the ApoE4 plus (ApoE4+) allele. In some specific embodiments, the rs28931579 SNP may comprise the nucleic acid sequence as denoted by SEQ ID NO. 40, that encode the amino acid sequence as denoted by SEQ ID NO. 41. According to such embodiment, the method may comprise the steps of contacting said cell with an effective amount of a Cas protein that specifically recognizes the 5′-NGCG-3′ PAM, specifically, 5′-GCGC-3′ PAM within the rs28931579 allele (ApoE4+), and with at least one nucleic acid sequence comprising at least one gRNA that targets a protospacer located upstream to said PAM within the rs28931579 allele. In some particular embodiments, such gRNA may comprise the nucleic acid sequence GTTAGTGACTTGCGGCTTC as denoted by SEQ ID NO. 31).

The methods of the invention provide targeted elimination of a pathogenic alleles, such as the ApoEε4 as well as the rs28931579 alleles. The term pathogenic, as used herein, relates to the ability of the specific allele to cause, produce or aggravate a disease or any pathologic disorder, or in some embodiments, associated with said disorders and conditions. It should be appreciated that in some embodiments, the ApoE4 and ApoE4+(s28931579) alleles may be associated with any of the disorders disclosed by the invention.

Still further, it should be appreciated that the invention may provide in some embodiments thereof targeted elimination of two or more pathogenic forms of the ApoE protein, for example, the ApoEε4 as well as the rs28931579 alleles (ApoE4+), using the Cas protein that specifically recognizes the 5′-NGCG-3′ PAM, specifically, the 5′-TCGC-3′ PAM within the ApoEε4 allele, as well as the 5′-GCGC-3′ PAM within the rs28931579 allele. According to such embodiments, for targeted elimination of both pathogenic alleles, at least one nucleic acid sequence comprising at least one gRNA that targets a protospacer located upstream to said PAM within the rs28931579 allele (ApoE4+), and at least one gRNA that targets a protospacer located upstream to said PAM within the ApoEε4 allele or any combinations thereof, may be used. Table 1 herein summarizes different properties of the ApoE variants discussed by the invention.

TABLE 1
Relevant alleles and SNPs of the ApoE locus

| | | | | SEQ ID NO: | |
|---|---|---|---|---|---|
| | SNP | Amino acid | | nucleic | amino |
| Genotype | reference | Substitution | PAM | acids | acids |
| ApoE2 | no SNP | Cys112/Cys158 | No | 38 | 39 |
| Also referred | | Also referred to as | | | |
| to as ApoEε2 | | Cys 130/Cys176 | | | |
| ApoE3 | rs7412 | Cys112/Arg158 | No | 1 | 2 |
| Also referred | | Also referred to as | | | |
| to as ApoEε3 | | Cys130/Arg176 | | | |
| ApoE4 | rs429358 and | Arg112/Arg158 | Yes | 3 | 4 |
| Also referred | rs7412 | Also referred to as | TGCG | | |
| to as ApoEε2 | | Arg130/Arg176 | | | |
| ApoE4(+) | rs28931579 | Ser296Arg | Yes | 40 | 41 |
| Also referred | | Also referred to as | GCGC | | |
| to as | | Ser314Arg | | | |
| rs28931579 | | | | | |

The invention in some embodiments thereof utilizes the CRISPR system for specific elimination of the ApoE4 protein. The Clustered Regularly Interspaced Short Palindromic Repeats (CRISPR) system is a bacterial immune system that has been modified for genome engineering.

CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

As used herein, CRISPR arrays also known as SPIDRs (Spacer Interspersed Direct Repeats) constitute a family of recently described DNA loci that are usually specific to a particular bacterial species. The CRISPR array is a distinct class of interspersed short sequence repeats (SSRs) that were first recognized in E. coli. In subsequent years, similar CRISPR arrays were found in Streptococcus pyogenes, Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly any of the CRISPR systems disclosed herein after. The CRISPR-Cas system has evolved in prokaryotes to protect against phage attack and undesired plasmid replication by targeting foreign DNA or RNA. The CRISPR-Cas system, targets DNA molecules based on short homologous DNA sequences, called spacers that exist between repeats. These spacers guide CRISPR-associated (Cas) proteins to matching (and/or complementary) sequences within the target DNA (e.g., foreign DNA), called proto-spacers, which are subsequently cleaved. The spacers can be rationally designed to target any target DNA sequence, for example, within the ApoE4 coding sequence.

The methods of the invention thus comprise the provision and use of gRNA that provides the specific targeting ability of the methods and compositions of the invention, specifically to the ApoE4 allele, or alternatively or additionally, the ApoE rs28931579 allele. In some specific embodiments, the exact specific targeting of the CRISPR system used by the invention to the ApoE4 coding sequence is further enhanced by the use of a specific PAM sequence that distinguish between the ApoE4 and ApoE3 coding sequences. In some specific embodiments, the nucleic acid sequence of ApoE3 is provided as SEQ ID NO. 1 and its amino acid sequence is provided as SEQ ID NO. 2.

As detailed above, in certain embodiments, the methods and compositions of the invention may use at least one cas protein, or alternatively, a nucleic acid sequence that comprise a sequence encoding said at least one Cas protein. In some embodiments, said encoding sequence may be referred to herein as a cas gene. As used herein, the term “cas gene” refers to the genes that are generally coupled, associated or close to or in the vicinity of flanking CRISPR arrays that encode Cas proteins.

In some embodiments, the cas protein may be a member of at least one of CRISPR-associated system Class 1 and/or Class 2. More specifically, at least one of Class 1, that may in some embodiments be divided into types I, III, and IV, and Class 2 that may be divided into types II, V, and VI. Thus, in certain embodiments, the Cas [protein applicable in the methods of the invention may be any Cas9 of type II, type I and type III, type IV, type V or type VI.

In some specific embodiment, the RNA guided DNA binding protein nuclease of the system of the invention may be a CRISPR Class 2 system. In yet some further particular embodiments, such class 2 system may be a CRISPR type II system.

In a more specific embodiment, the RNA guided DNA binding protein nuclease may be CRISPR-associated endonuclease 9 (Cas9) system.

The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein. However, as the HNH nuclease domain is abundant in restriction enzymes and possesses endonuclease activity, it is likely to be responsible for target cleavage. Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. This cleavage is catalyzed by the housekeeping, double-stranded RNA-specific RNase III in the presence of Cas9. Still further, it should be noted that type II system comprise cas9 and optionally, at least one of cas1, cas2 csn2, and cas4 genes. It should be appreciated that any type II CRISPR-Cas systems may be applicable in the present invention, specifically, any one of type II-A or B.

In certain embodiments, it should be understood that any cas member of the type II CRISPR system may be applicable in the invention.

Thus, in yet some specific embodiments, the at least one cas gene used in the methods and compositions of the invention may be at least one cas gene of type II CRISPR system (either typeII-A or typeII-B). In more particular embodiments, at least one cas gene of type II CRISPR system used by the invention may be the cas9 gene. It should be appreciated that such system may further comprise at least one of cas1, cas2, csn2 and cas4 genes.

Double-stranded DNA (dsDNA) cleavage by Cas9 is a hallmark of “type II CRISPR-Cas” immune systems. The CRISPR-associated protein Cas9 is an RNA-guided DNA endonuclease that uses RNA:DNA complementarity to identify target sites for sequence-specific double stranded DNA (dsDNA) cleavage. CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion of the endogenous tracrRNA with a targeting sequence (also named crRNA), providing both scaffolding/binding ability for Cas9 nuclease and targeting specificity. Also referred to as “single guide RNA” or “sgRNA”. The targeted DNA sequences are specified by the CRISPR array, which is a series of 30-40 bp spacers separated by short palindromic repeats. The array is transcribed as a pre-crRNA and is processed into shorter crRNAs that associates with trans-activating crRNA to form the guide RNA provided by the kit of the invention separately. The gRNA associates with the Cas protein complex to target complementary DNA sequences known as proto-spacers. These proto-spacer targets in the genomic DNA must also have an additional neighboring sequence known as a proto-spacer adjacent motif (PAM) that is required for target recognition. After binding, a Cas protein complex serves as a DNA endonuclease to cut both strands at the target and subsequent DNA degradation occurs via exonuclease activity. Once expressed, the Cas9 protein and the gRNA provided by the invention form a riboprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex binds any target genomic sequence with a PAM, but the extent to which the gRNA spacer matches the target DNA determines whether Cas9 will cut, or alternatively, perform any other manipulation in case a fusion protein comprising a catalytically inactive cas9 is used (for example, by the diagnostic method of the invention). Once the Cas9-gRNA complex binds a DNA target, a “seed” sequence at the 3′ end of the gRNA targeting sequence begins to anneal to the target DNA. If the seed and target DNA sequences match, the gRNA continues to anneal to the target DNA in a 3′ to 5′ direction.

In yet some further embodiments it should be understood that any of type of a CRISPR system of any class may be applicable for the methods of the invention, particularly, any one of type II, type I and type III, type IV, type V or type VI.

In more specific embodiments, the methods and systems of the invention may use Type I and Type III.

More specifically, Type I CRISPR-Cas systems contain the cas3 gene, which encodes a large protein with separate helicase and DNase activities, in addition to genes encoding proteins that probably form Cascade-like complexes with different compositions. These complexes contain numerous proteins that have been included in the repeat-associated mysterious proteins (RAMPs), which form a large superfamily of Cas proteins, and contain at least one RNA recognition motif (RRM; also known as a ferredoxin-fold domain) and a characteristic glycine-rich loop. RAMP superfamily encompasses the large Cas5 and Cas6 families on the basis of extensive sequence and structure comparisons. Furthermore, the Cas7 (COG1857) proteins represent another distinct, large family within the RAMP superfamily. The type I CRISPR-Cas systems seem to target DNA where the target cleavage is catalyzed by the HD nuclease domains of Cas3. As the RecB nuclease domain of Cas4 is fused to Cas1 in several type I CRISPR-Cas systems, Cas4 could potentially play a part in spacer acquisition instead. It should be noted that any type I CRISPR-Cas systems may be applicable in the present invention, specifically, any one of type I-A, B, C, D, E, and F.

The type III CRISPR-Cas systems contain polymerase and RAMP modules in which at least some of the RAMPs seem to be involved in the processing of the spacer-repeat transcripts, analogous to the Cascade complex. Type III systems can be further divided into sub-types III-A (also known as Mtube or CASS6) and III-B (also known as the polymerase-RAMP module). Subtype III-A systems can target plasmids, as has been demonstrated in vivo for S. epidermidis, and it seems plausible that the HD domain of the polymerase-like protein encoded in this subtype (COG1353) might be involved in the cleavage of target DNA. It should be appreciated that any cas gene that belongs to the type III CRISPR system may be used for the purpose of the invention, for example, any one of cas6, cas10, csm2, csm3, csm4, csm5, csm6, cmr1, cmr3, cmr4, cmr5, cmr6, cas1 and cas2. Still further, any one of typeIII-A or typeIII-B systems may be used for the kits and method of the invention.

In yet some specific and non-limiting embodiments, the cas protein used by the methods of the invention may be CRISPR associated protein 9 (cas9) variant that specifically recognizes the 5′-NGCG-3′ PAM, or any derivative or fusion protein thereof.

In some particular embodiments, a Cas9 variant that specifically recognizes the NGCG PAM, may be a Cas9 variant that carry at least one of substitution in residues 1135, 1218, 1335 and 1335 of the Cas 9 protein, specifically, the spCas9, that may comprise in some embodiments the amino acid sequence as denoted by SEQ ID NO. 28, encoded by the nucleic acid sequence as denoted by SEQ ID. NO. 29. In some specific and non-limiting embodiments, such variant may comprise at least one of, Valine at residue 1135 (1135V) of the spCas9 sequence, specifically, as denoted by SEQ ID NO. 28, Arginine at residue 1218 of the spCas9 sequence, specifically, as denoted by SEQ ID NO. 28, Glycine at residue 1335 of the spCas9 sequence, specifically, as denoted by SEQ ID NO. 28 (1335G), and Arginine at residue 1337 of the spCas9 sequence, specifically, as denoted by SEQ ID NO. 28 (1337R). Thus, in some specific and non-limiting embodiments, such variant may comprise at least one of the D1135V, G1218R, R1335G and T1337R substitutions. In some particular embodiment, the cas9 variant may comprise at least one of Asp1135 Val (D1135V), Glu1218 to Arg (G1218R), Arg1335Glu (R1335G) and Thr1337Arg (T1337R) substitutions.

In yet some further specific embodiments, the cas9 variant may be the Streptococcus pyogenes Cas9 (SpCas9) VRER variant or any derivative or fusion protein thereof.

In certain specific embodiments, the SpCas9 VRER variant may comprise the amino acid sequence as denoted by SEQ ID NO. 6.

Thus, in some embodiments, the methods of the invention may comprise the step of providing an effective amount of a Cas9 variant that comprises the amino acid sequence as denoted by SEQ ID NO. 6. In yet some alternative embodiments, the methods of the invention may provide a nucleic acid molecule that comprises a nucleic acid sequence encoding the Cas9 variant that comprises the amino acid sequence as denoted by SEQ ID NO. 6. In some particular embodiments, such nucleic acid sequence may comprise a sequence as denoted by SEQ ID NO. 5.

As noted above, the type II cas protein used by the invention may be the Cas9. However, it should be appreciated that any cas protein of type II CRISPR system may be applicable in the invention.

In yet some further alternative embodiments, the targeted destruction of ApoE4 may be performed using the nuclease Cpf1 from the CRISPR/Cpf1 system of the bacterium Francisella novicida. Cpf1 is classified as a Cas type V enzyme. Cpf1 showed several key differences from Cas9 including: causing a ‘staggered’ cut in double stranded DNA as opposed to the ‘blunt’ cut produced by Cas9, relying on a ‘T rich’ PAM (providing alternate targeting sites to Cas9) and requiring only a CRISPR RNA (crRNA) for successful targeting. By contrast Cas9 requires both crRNA and a transactivating crRNA (tracrRNA).

It should be noted that the cas protein used by the methods and compositions of the invention may be provided either as a polypeptide, or alternatively, as will be discussed herein after, may be provided as nucleic acid sequence encoding said polypeptide.

The term “polypeptide” as used herein refers to amino acid residues, connected by peptide bonds. A polypeptide sequence is generally reported from the N-terminal end containing free amino group to the C-terminal end containing free carboxyl group. More specifically, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

As noted above, the VRER cas9 variant used by the methods and compositions of the invention may be the cas9 comprising the amino acid sequence as denoted by SEQ ID NO. 6, or any derivative, variant of fusion protein comprising the same. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present invention.

Thus, in some embodiments, the methods and compositions of the invention and particularly, the polypeptide and any polynucleotide encoding the polypeptide in accordance with the present invention applies to a plurality of CRISPR-cas proteins orthologs or homologues having a sequence homology or identity to the cas proteins used as described herein after, of at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the Cas9 variant as denoted by SEQ ID NO. 6. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 6, specifically, the entire sequence as denoted by SEQ ID NO. 6.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides used by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to peptide, polypeptide, or protein sequence thereby altering, adding or deleting a single amino acid or a small percentage of amino acids in the encoded sequence is a “conservatively modified variant”, where the alteration results in the substitution of an amino acid with a chemically similar amino acid.

Conservative substitution tables providing functionally similar amino acids are well known in the art. Such conservatively modified variants are in addition to and do not exclude polymorphic variants, interspecies homologues, and alleles and analogous peptides of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

The derivatives of any of the polypeptides according to the present invention, e.g. of a specified sequence of any Cas9 variant that specifically recognizes the NGCG PAM, specifically, a Cas9 variant that carry at least one substitution is residues 1135, 1218, 1335 and 1335 of the Cas 9 protein (specifically, the spCas9, that may comprise in some embodiments the amino acid sequence as denoted by SEQ ID NO. 28, encoded by the nucleic acid sequence as denoted by SEQ ID. NO. 29), specifically, at least one of the D1135V, G1218R, R1335G and T1337R substitutions, more specifically, the polypeptide of SEQ ID NOs: 6, may vary in their size and may comprise the full length polypeptide or any fragment thereof that sufficiently retaining the function of recognizing and binding the specific PAM 5′-NGCG-3′, specifically in the ApoE gene, more specifically, in the ApoE4 allele. In some embodiments, the derivatives may include modified amino acid residues.

In yet some further embodiments, particularly if provided as a protein product, the Cas9 used by the invention can be coupled (conjugated) through any of their residues to another peptide or agent. For example, the polypeptides of the invention can be coupled through their N-terminus to a lauryl-cysteine (LC) residue and/or through their C-terminus to a cysteine (C) residue.

Further, the peptides may be extended at the N-terminus and/or C-terminus thereof with various identical or different amino acid residues. As an example for such extension, the peptide may be extended at the N-terminus and/or C-terminus thereof with identical or different amino acid residue/s, which may be naturally occurring or synthetic amino acid residue/s. It must be appreciated that the description herein that relates to variants, derivatives and homologs applies to any of the amino acid and/or nucleic acid sequences disclosed by the invention.

The second element provided by the methods of the invention may be a specific gRNA that specifically targets the ApoE4 coding sequence, or in alternative embodiments, the rs28931579 allele, as also described by Table 1, herein before. According to some embodiments, the polynucleotide encoding the gRNA of the invention may comprise at least one spacer and optionally, at least one repeat. In yet some further embodiments, the DNA encoding the gRNA of the invention may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200 or more spacers. In some embodiments, each spacer is located between two repeats.

As used herein, the term “spacer” refers to a non-repetitive spacer sequence that is designed to target a specific sequence and is located between multiple short direct repeats (i.e., CRISPR repeats) of CRISPR arrays. In some specific embodiments, spacers may comprise between about 15 to about 30 nucleotides, specifically, about 15, 16, 17, 18, 19, 20, 21, 22, 23, 24, 25, 26, 27, 28, 29, 30 or more nucleotides. More specifically, about 20-25 nucleotides.

The guide or targeting RNA encoded by the CRISPR system of the invention may comprise a CRISPR RNA (crRNA) and a trans activating RNA (tracrRNA). The sequence of the targeting RNA encoded by the CRISPR spacers, is not particularly limited, other than by the requirement for it to be directed to (i.e., having a segment that is the same as or complementarity to) a target sequence in a genomic DNA that is also referred to herein as a “proto-spacer”, specifically within the ApoE4 coding sequence. Such proto-spacers comprise nucleic acid sequence having sufficient complementarity to a targeting RNA encoded by the CRISPR spacers comprised within the nucleic acid sequence encoding the gRNA of the invention.

In some embodiments, a crRNA comprises or consists of 20, 21, 22, 23, 24, 25, 26, 27, 28, 29, 30, 31, 32, 33, 34, 35, 36, 37, 38, 39 or 40 nucleotides of the spacer (targeting) sequence followed by 19-36 nt of repeat sequence. In specific and non-limiting embodiments, the targeting spacer may comprise or consist of a segment that targets any genomic DNA sequence of ApoE4, for which a representative spacer sequences is indicated herein.

It should be noted that in some embodiments, the spacers of the CRISPR system of the invention may encode a targeting guide RNA (gRNA).

A “gRNA” or “targeting RNA” is an RNA that, when transcribed from the portion of the CRISPR system encoding it, comprises at least one segment of RNA sequence that is identical to (with the exception of replacing T for U in the case of RNA) or complementary to (and thus “targets”) a DNA sequence in the target genomic DNA, specifically, within the ApoE4 gene.

In some embodiments, the crRNA is a single-stranded ribonucleic acid (ssRNA) sequence complementary to a target genomic DNA sequence that is also disclosed herein as a protospacer. More specifically, the target genomic DNA sequence is located in some embodiments, upstream of a 5′-TGCG-3′ PAM sequence within the ApoEε4 allele, or alternatively, upstream of a 5′-GGCG-3′ PAM sequence within the ApoE4+ allele. It should be noted that the terms used herein “upstream” and “downstream” both refer to a relative position in DNA or RNA. Each strand of DNA or RNA has a 5′ end and a 3′ end, so named for the carbon position on the deoxyribose (or ribose) ring. By convention, upstream and downstream relate to the 5′ to 3′ direction in which RNA transcription takes place. Upstream is toward the 5′ end of the DNA or RNA molecule and downstream is toward the 3′ end. When considering double-stranded DNA, upstream is toward the 5′ end of the protein coding strand for the gene in question and downstream is toward the 3′ end. Due to the anti-parallel nature of DNA, this means the 3′ end of the mRNA template strand is upstream of the gene and the 5′ end is downstream.

As used herein, the term “5′” refers to the part of the strand that is closer to the 5′ end or 5′ terminus, i.e. to the extremity of the DNA or RNA strand that has a phosphate group attached to the fifth carbon in the sugar-ring of the deoxyribose or ribose at its terminus. Furthermore, the term “3′” refers to the part of the strand that is closer to the 3′ end or 3′ terminus, i.e. to the extremity of the DNA or RNA strand that has a hydroxyl group linked to the 3rd carbon in the sugar-ring of the deoxyribose or ribose at its terminus.

In addition, in order to define the position of a nucleotide on a DNA coding strand, the terms “minus” (also represented by the “−” symbol) or “plus” (also represented by the “+” symbol) are employed. The term “minus” corresponds to a position which is upstream to the Transcription Start Site-TSS (considered as the position “one”) and the term “plus” corresponds to a position which is downstream to the TSS.

In some embodiments, the gRNA used by the methods of the invention may target a protospacer comprising the nucleic acid sequence as denoted by SEQ ID NO. 18 of the ApoEε4 allele, or any fragments thereof. More specifically, in some further embodiments, the gRNA sequence of the invention may target a sequence comprising the sequence GGCGCGGACATGGAGGAC (also denoted by SEQ ID NO. 18), and in some embodiments, such targeted protospacer sequence may be almost identical to the sequence of the gRNA, specifically, GGGCGCGGACATGGAGGAC as denoted by SEQ ID NO. 8. In some embodiments, the protospacer of SEQ ID NO. 18 is complementary to the sequence of SEQ ID NO. 30. In yet some further embodiments, the protospacer sequence may comprise the exact sequence of the gRNA, specifically, as denoted by SEQ ID NO. 8. It should be noted that such sequence is located 5′ to the PAM TGCG, recognized by the Cas9 variant used by the invention, as also illustrated by FIG. 2.

In yet an alternative embodiment, where the rs28931579 SNP (ApoE4+) is targeted by the methods of the invention, the gRNA used by the methods of the invention may target a protospacer comprising the nucleic acid sequence as denoted by SEQ ID NO. 36, or any fragments thereof. In some embodiments, such targeted protospacer sequence may be identical to the sequence of the gRNA, specifically, GCTTCGGCGTTCAGTGATTGT as denoted by SEQ ID NO. 31 (it should be noted that this sequence is presented from 5′ to 3′, however in FIG. 9 the sequence is represented from 3′ to 5′). More specifically, in some further embodiments, the gRNA sequence of the invention may target a sequence that is complementary to the sequence of SEQ ID NO. 37.

As indicated herein, the gRNA of the invention may be complementary, at least in part, to the target genomic DNA, specifically, the target protospacer in ApoE4 coding sequence. In certain embodiments, “Complementarity” refers to a relationship between two structures each following the lock-and-key principle. In nature complementarity is the base principle of DNA replication and transcription as it is a property shared between two DNA or RNA sequences, such that when they are aligned antiparallel to each other, the nucleotide bases at each position in the sequences will be complementary (e.g., A and T or U, C and G). As indicated above, the genomic DNA sequence targeted by the gRNA of the kit of the invention is located immediately upstream to a PAM sequence. In some embodiments, such PAM sequence may be of the nucleic acid sequence NGCG. In certain embodiments, the PAM sequence referred to by the invention may comprise N, that is any nucleotide, specifically, any one of Adenine (A), Guanine (G), Cytosine (C) or Thymine (T). In yet some further embodiments the PAM sequence according to the invention is composed of A, G, C, or T, followed by a Guanine, followed by a Cytosine and a Guanine. In more specific embodiments, the PAM sequence referred to by the invention in connection with the ApoE4 allele, comprises Thymine (T), followed by a Guanine, followed by a Cytosine and a Guanine. In yet some further specific embodiments, the PAM sequence referred to by the invention in connection with the ApoE4+ allele, comprises Guanine (G), followed by a Guanine, followed by a Cytosine and a Guanine.

It should be noted that the specific PAM sequence TGCG that is specifically recognized by the VRER cas9 variant, distinguish between the ApoE4 and ApoE3 isoforms and serves as an additional tool by the methods of the invention to increase specificity to the ApoE4 allele.

Still further, in some specific embodiments, to increase and determine specificity, a gRNA that specifically targets a protospacer located upstream to the specific PAM is used. Such gRNA, in some embodiments, may be an sgRNA. In more specific embodiments, for targeting the ApoE4 allele a gRNA that may comprise the nucleic acid sequence as denoted by SEQ ID NO. 8, may be used. In some alternative embodiments, the methods and compositions of the invention may use gRNA comprising the nucleic acid sequence as denoted by any one of SEQ ID NO. 19, 20, 21 or 22, or any combinations thereof. In other specific embodiments, for targeting the ApoE4+ allele a gRNA that may comprise the nucleic acid sequence as denoted by SEQ ID NO. 31, may be used.

As noted above, the methods and compositions of the invention involves the use of the Cas protein and the gRNA or any nucleic acid encoding the same. As used herein, “nucleic acids” is interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. The term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may range from about 8 to about 1,000 nucleotides long. More specifically, the oligonucleotide molecule/s used by the methods and compositions of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 200, 300, 400, 500, 600, 700, 800, 900, 1000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000 or more bases in length.

In some specific embodiments, in cases where at least one component used by the invention, specifically, at least one of, the polypeptide and the gRNA, may be provided as a nucleic acid sequence encoding said polypeptide or gRNA, such encoding nucleic acid sequence may be provided comprised within a vector. Such vector may be in certain embodiments, any plasmid, construct, phagemid, an engendered bacteriophage or viral vector comprising the encoding nucleic acid sequence described herein. Such vectors or constructs may be also referred to herein as recombinant nucleic acids. As used herein, the term “recombinant DNA”, “recombinant nucleic acid sequence” or “recombinant gene” refers to a nucleic acid comprising an open reading frame encoding one of the polypeptide, specifically the cas protein or alternatively, a nucleic acid sequence encoding the gRNA of the invention. In yet some further embodiments, the gRNA of the invention may be provided as an gRNA molecule.

Thus, in some embodiments, the polypeptide (e.g., Cas9) or gRNA encoding sequences may be provided in any vector. In some embodiments, such vector may also comprise in addition to at least one nucleic acid sequence encoding at least one gRNA in accordance with the invention, also nucleic acid sequence encoding the Cas9 variant used by the invention (specifically, any Cas9 that recognizes the PAM of the invention). The invention thus further relates to recombinant DNA constructs comprising the polynucleotides of the invention, that may in some embodiments comprise nucleic acid sequences encoding the at least one gRNA, the Cas9, or both. Such constructs may optionally further comprise additional elements such as promoters, regulatory and control elements, translation, expression and other signals, operably linked to the nucleic acid sequence of the invention.

The phrase “operatively-linked” is intended to mean attached in a manner which allows for transgene transcription. The term “encoding” is intended to mean that the subject nucleic acid may be transcribed and translated into either the desired polypeptide or the subject protein in an appropriate expression system, e.g., when the subject nucleic acid is linked to appropriate control sequences such as promoter and enhancer elements in a suitable vector (e.g., an expression vector) and when the vector is introduced into an appropriate system or cell. “Vectors” or “Vehicles”, as used herein, encompass vectors such as plasmids, phagemides, viruses, bacteriophage, integratable DNA fragments, and other vehicles, which enable the integration of DNA fragments into the genome of the host, or enable expression of genetic elements that are not integrated. Vectors are typically self-replicating DNA or RNA constructs containing the desired nucleic acid sequences, and operably linked genetic control elements that are recognized in a suitable host cell and effect the translation of the desired spacers. Generally, the genetic control elements can include a prokaryotic promoter system or a eukaryotic promoter expression control system. Such system typically includes a transcriptional promoter and transcription enhancers to elevate the level of RNA expression. Vectors usually contain an origin of replication that allows the vector to replicate independently of the host cell. Accordingly, the term control and regulatory elements includes promoters, terminators and other expression control elements. Such regulatory elements are described in the art and known to the skilled artisan. For instance, any of a wide variety of expression control sequences that control the expression of a DNA sequence when operatively linked to it may be used in these vectors to express DNA sequences encoding any desired cas protein and gRNA using the method of this invention.

A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein.

In some specific and non-limiting embodiments, the Cas9 protein may be provided by the MSP1101 plasmid (comprising nucleic acid sequence as denoted by SEQ ID NO 7, Addgene, plasmid cat. No. #65773). It yet some further specific and non-limiting embodiment, the gRNA of the invention may be provided using the Lenti_sgRNA_EFS_GFP (LRG) plasmid (Addgene, plasmid cat. No. #65656). It must be understood that in some embodiments, the gRNA of the invention may be provided either alone in a separate vector, as discussed above, or alternatively, in a vector that comprises nucleic acid sequence encoding the sgRNA of the invention together with a nucleic acid sequence encoding the Cas9 variant of the invention, using a the Lenti-viral plasmid (Addgene, plasmid cat. No. #52961). A non-limiting example for such lentiviral vector that comprises sequences encoding the gRNA and the Cas9 variant of the invention may be a vector that comprises the nucleic acid sequence as denoted by SEQ ID NO. 27, or any derivatives or homologs thereof.

It should be appreciated that the methods and compositions of the invention and particularly, the polynucleotides of the invention further encompass sequences having homology of at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the vectors as denoted by SEQ ID NO. 27, or SEQ ID NO. 7. Specifically, homologs that comprise or consists of a nucleic acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 27 or SEQ ID NO. 7, specifically, the entire sequence as denoted by SEQ ID NOs. 27 or 7.

It should be appreciated that in certain embodiments, the oligonucleotide/s or polynucleotide/s used by the method/s and compositions of the invention are isolated and/or purified molecules. As used herein, “isolated” or “purified” when used in reference to a nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95% pure) of non-nucleotide material naturally associated with it, and thus is distinguished from isolated chromosomes. As used herein, the terms “isolated” and “purified” in the context of a proteinaceous agent (e.g., a peptide, polypeptide, protein or antibody) refer to a proteinaceous agent which is substantially free of cellular material and in some embodiments, substantially free of heterologous proteinaceous agents (i.e. contaminating proteins) from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous proteinaceous agent (e.g. protein, polypeptide, peptide, or antibody; also referred to as a “contaminating protein”). When the proteinaceous agent is recombinantly produced, it is also preferably substantially free of culture medium, i.e. culture medium represents less than about 20%, 10%, or 5% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. Preferably, proteinaceous agents disclosed herein are isolated.

In some specific embodiments, the method of the invention may be particularly useful for targeted elimination of the ApoE4 in a subject that carry at least one ApoEε4 allele.

In some embodiments, subjects that carry at least one ApoEε4 allele, may be heterozygotes that carry the ApoEε4 allele and the ApoEε3 allele or heterozygotes that carry the ApoEε2 allele and the ApoEε4 allele (ε3/ε4 or ε2/ε4, respectively), as well as homozygotes that carry the ApoEε4 at both alleles (ε4/ε4). Specific definition of these alleles is disclosed in Table 1 herein before.

The term “homozygous” is employed for a particular gene when identical alleles of the gene are present on both homologous chromosomes. The cell or organism in question is called a homozygote.

The term “heterozygous” relates to a gene locus with two different alleles of a gene. The cell or organism is called a heterozygote specifically for the allele in question.

As indicated above, ApoE4 isoform has been implicated in variety of pathologic conditions, and thereof specific elimination thereof provides a specific therapeutic tool for treating and preventing disorders or conditions caused thereby.

Thus, in a further aspect, the invention provides a method for treating, preventing, ameliorating, inhibiting or delaying the onset of a pathologic condition or disease associated with at least one pathogenic form of the Apo E protein in a mammalian subject.

More specifically, the method of the invention comprises the step of administering a therapeutically effective amount of: (a) at least one polypeptide comprising at least one Cas protein, or any nucleic acid encoding said polypeptide, wherein said Cas protein specifically recognizes the 5′-NGCG-3′ PAM; and (b) at least one nucleic acid sequence comprising at least one gRNA that targets a protospacer located upstream to said PAM within the pathogenic ApoE allele, or any nucleic acid sequence encoding said gRNA. Alternatively, a construct, vehicle, vector, kit or composition comprising (a) and (b) may be also used by the method of the invention. In yet some further specific embodiments, the invention provides treating, preventing, ameliorating, inhibiting or delaying the onset of an ApoE4 associated pathologic condition or disease in a mammalian subject. More specifically, the method of the invention comprises the step of administering a therapeutically effective amount of: (a) at least one polypeptide comprising at least one Cas protein, or any nucleic acid encoding said polypeptide, wherein said Cas protein specifically recognizes the 5′-NGCG-3′ PAM, specifically, the 5′-TGCG-3′ PAM; and (b) at least one nucleic acid sequence comprising at least one gRNA that targets a protospacer located upstream to said PAM within the ApoEε4 allele, or any nucleic acid sequence encoding said gRNA. Alternatively, a kit or composition comprising (a) and (b) may be also used by the method of the invention.

In some further specific embodiments, the invention provides a method for treating, preventing, ameliorating, inhibiting or delaying the onset of an rs28931579 allele (ApoE4+) associated pathologic condition or disease in a mammalian subject. More specifically, the method of the invention may comprise the step of administering a therapeutically effective amount of: (a) at least one polypeptide comprising at least one Cas protein that recognizes the 5′-NGCG-3′ PAM specifically, the 5′-GGCG-3′ PAM, or any nucleic acid encoding said polypeptide; and (b) at least one nucleic acid sequence comprising at least one gRNA that targets a protospacer located upstream to said PAM within the rs28931579 allele, or any nucleic acid sequence encoding said gRNA; or a kit, construct, vector, vehicle or composition comprising (a) and (b).

It should be noted that an apoE4 driven brain pathology in accordance with the invention may include: (a) AD related pathologies (i.e., the accumulation of Abeta and hypephosphorylated tau in hippocampal synapses; (b) synaptic pathologies (i.e., decreased levels of the presynaptic marker synaptophysin and of the presynaptic glutamatergic and GABAergic transporters Vglut and Vgat and of the levels of the apoE receptor apoER2); (c) Diabetes related pathologies includes decrease levels of brain insulin receptors and impaired brain insulin metabolism. In addition since apoE4 is hypolipidated relative to the other apoE isoforms the method of the invention may further be applied in decreasing the level of the hypolipidated apoE4 form of apoE and thereby increasing the overall lipidation of apoE4.

Thus, as used herein the term apoE4-related disorders comprise neurodegenerative diseases such as Alzheimer's disease, Parkinson's disease, glaucoma, amyotrophic lateral sclerosis, dementia, Vascular dementia hyperalgesia states and any neurodegeneration as well as vascular conditions that may include in some embodiments, atherosclerosis and peripheral vascular diseases, as well as cardiovascular diseases such as coronary artery diseases (CAD) such as angina and myocardial infarction (commonly known as a heart attack), stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis, as well as Traumatic Brain Injury (TBI).

In some embodiments, the ApoE4 associated condition or disease treated by the invention may be at least one of an acute or chronic neurodegenerative, vascular, and inflammatory pathology or condition or any combination thereof.

As disclosed herein above, the methods of the invention are applicable in treating neurodegenerative disorders. Neurodegeneration is the umbrella term for the progressive loss of structure or function of neurons, including synaptic dysfunction and death of neurons. Many neurodegenerative diseases including Parkinson's and Alzheimer's are associated with neurodegenerative processes. Other examples of neurodegeneration that may be also applicable herein may include Friedreich's ataxia, Lewy body disease, spinal muscular atrophy, multiple sclerosis, frontotemporal dementia, corticobasal degeneration, progressive supranuclear palsy, multiple system atrophy, hereditary spastic paraparesis, amyloidosis, Amyotrophic lateral sclerosis (ALS), and Charcot Marie Tooth. It should not be overlooked that normal aging processes include progressive neurodegeneration, specifically, age-related cognitive decline (ACD) and mild cognitive impairment (MCI) are also applicable in the present invention.

Still further, it should be appreciated that the invention provides methods for treating or preventing any neuro-pathological condition. The term “neuro-pathological condition” relates to any pathological condition caused by, or which causes, or is associated with neural cell disorders, such as any deterioration of the neural cell functions or viability. Such conditions may be neurodegenerative disorders, ischemic and vascular diseases, brain traumas and neuronal inflammation. However, it should be appreciated that in some embodiments, any other disorders that involve neuronal degeneration may be also applicable in the present invention. More specifically, metabolic disorders which affect the nervous system, such as diabetes and phenylketonuria, immunological disorders which affect the brain, such as Hashimoto's Thyroiditis, genetic diseases which affects neural cells, such as Tay-Sachs disease, metachromatic leukodystrophy, Krabbe disease, Fabry disease, Gaucher disease, Farber disease, and Niemann-Pick disease, nutrient deficiencies such as vitamin B6 and D deficiencies, and any sequelae which affects the nervous system.

It should be further appreciated that the methods and compositions of the invention may be applicable for treating neuro-pathological and neurodegenerative disorders or of any pathologic condition associated therewith. It is understood that the interchangeably used terms “associated”, linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. Such conditions may include for example, Parkinson's disease, Alzheimer's disease, Down syndrome, head trauma, epilepsy, stroke, neuromyotonia/Isaacs syndrome, lower motor neuron lesion, Werdnig-Hoffman disease, Kennedy disease, subarachnoid hemorrhage, intracerebral hemorrhage, occlusion and stenosis of precerebral arteries, occlusion and stenosis of basilar artery, occlusion and stenosis of carotid artery, occlusion and stenosis of vertebral artery, occlusion of cerebral arteries, cerebral thrombosis with or without cerebral infarction, cerebral embolism with or without cerebral infarction, transient cerebral ischemia, basilar artery syndrome, vertebral artery syndrome, subclavian steal syndrome, vertebrobasilar artery syndrome, transient ischemic attack (TIA), cerebral atherosclerosis, hypertensive encephalopathy, cerebral aneurysm, cerebral arteritis, Moyamoya Disease, nonpyogenic thrombosis of intracranial venous sinus, atherosclerosis, atherosclerosis of renal artery, atherosclerosis of native arteries of the extremities, intermittent claudication, aortic aneurysm, dissection of aorta, dissection of carotid artery, dissection of iliac artery, dissection of renal artery, dissection of vertebral artery, erythromelalgia, and polyarteritis nodosa.

The apoE4 allele is the strongest known genetic risk factor for Alzheimer's Disease which the most common form of age-related dementia and is characterized by neuronal degeneration, synaptic loss, brain atrophy and inflammation as well as accumulation of insoluble aggregates of amyloid β and tau proteins. The ApoE protein has been implicated in many of the processes.

ApoE-lipoproteins bind to several cell-surface receptors to deliver lipids and also to hydrophobic amyloid-β (Aβ) peptide, which is thought to initiate toxic events that lead to synaptic dysfunction and neurodegeneration in AD. ApoE isoforms differentially regulate Aβ aggregation and clearance in the brain, and have distinct functions in regulating brain lipid transport, glucose metabolism, neuronal signaling, neuroinflammation, and mitochondrial function.

A group of disorders associated with beta-amyloid protein aggregation include Alzheimer's disease (AD), where deposits of a protein precursor called beta-amyloid build up (termed plaques) in the spaces between nerve cells and twisted fibers of tau protein build up (termed tangles) inside the cells.

Thus, in some embodiments, ApoE4 associated disorders may include any pathologic condition involving Beta-amyloid protein aggregations. More specifically, “Beta-amyloid protein aggregations” as used herein relates to cerebral plaques laden with β-amyloid peptide (Aβ) and dystrophic neurites in neocortical terminal fields as well as prominent neurofibrillary tangles in medial temporal-lobe structures, which are important pathological features of Alzheimer's disease. Subsequently, loss of neurons and white matter, congophilic (amyloid) angiopathy are also present.

Aβ peptides are natural products of metabolism consisting of 36 to 43 amino acids. Monomers of Aβ40 are much more prevalent than the aggregation-prone and damaging Aβ42 species. β-amyloid peptides originate from proteolysis of the amyloid precursor protein by the sequential enzymatic actions of beta-site amyloid precursor protein-cleaving enzyme 1 (BACE-1), a β-secretase, and γ-secretase, a protein complex with presenilin 1 at its catalytic core. An imbalance between production and clearance, and aggregation of peptides, causes Aβ to accumulate, and this excess may be the initiating factor in Alzheimer's disease.

β-amyloid can also grow into fibrils, which arrange themselves into β-pleated sheets to form the insoluble fibers of advanced amyloid plaques. Soluble oligomers and intermediate amyloid are the most neurotoxic forms of Aβ. In brain-slice preparations, dimers and trimers of Aβ are toxic to synapses. Experimental evidence indicates that Aβ accumulation precedes and drives tau protein aggregation.

It should be further appreciated that ApoE4 associated disorders may also include any condition associated with Tau protein aggregation. “Tau protein” as used herein, refers to neurofibrillary tangles, which are filamentous inclusions in pyramidal neurons, characteristic for Alzheimer's disease and other neurodegenerative disorders termed tauopathies. Elucidation of the mechanisms of their formation may provide targets for future therapies. Accumulation of hyperphosphorylated Tau protein as paired helical filaments in pyramidal neurons is a major hallmark of Alzheimer disease. Besides hyperphosphorylation, other modifications of the Tau protein, such as cross-linking, are likely to contribute to the characteristic features of paired helical filaments, including their insolubility and resistance against proteolytic degradation. These neurofibrillary tangles, consist of hyperphosphorylated and aggregated forms of the microtubule-associated protein tau.

Under non-pathological conditions, tau is a developmentally regulated phosphoprotein that promotes assembly and stability of microtubules and is thus involved in axonal transport. In AD and other tauopathies, tau proteins aggregate and form fibrillar insoluble intracellular inclusions, so-called neurofibrillary tangles. It has been suggested that ionic interactions and covalent cross-linking contribute to pathological Tau aggregation and tangle formation. Reactive carbonyl compounds, which are increased under conditions of oxidative stress and in aging have been proposed as potential compounds responsible for tau aggregation.

The terms “inflammatory disease” or “inflammatory-associated condition” refers to any disease or pathologically condition which can benefit from the reduction of at least one inflammatory parameter, for example, induction of an inflammatory cytokine such as IFN-gamma and IL-2 and reduction in IL-6 levels. The condition may be caused (primarily) from inflammation, or inflammation may be one of the manifestations of the diseases caused by another physiological cause.

In some specific embodiments, ApoE4 associated disorder may be Alzheimer's disease. It should be understood that in some specific embodiments, such chronic neurodegenerative disorder may further involve inflammatory and/or vascular causes.

More specifically, “Alzheimer's disease (AD)”, as used herein refers to a disorder that involves deterioration of memory and other cognitive domains that in general leads to death within 3 to 9 years after diagnosis. The principal risk factor for Alzheimer's disease is age. The incidence of the disease doubles every 5 years after 65 years of age. Up to 5% of people with the disease have early onset AD (also known as younger-onset), that may appear at 40 or 50 years of age. Many molecular lesions have been detected in Alzheimer's disease, but the overarching theme to emerge from the data is that an accumulation of misfolded proteins in the aging brain results in oxidative and inflammatory damage, which in turn leads to energy failure and synaptic dysfunction. Alzheimer's disease may be primarily a disorder of synaptic failure. Hippocampal synapses begin to decline in patients with mild cognitive impairment (a limited cognitive deficit often preceding dementia) in whom remaining synaptic profiles show compensatory increases in size. In mild Alzheimer's disease, there is a reduction of about 25% in the presynaptic vesicle protein synaptophysin. With advancing disease, synapses are disproportionately lost relative to neurons, and this loss is the best correlate with dementia. Aging itself causes synaptic loss, which particularly affects the dentate region of the hippocampus.

There is no single linear known chain of events or pathways that could initiate and drive Alzheimer's disease. AD is a progressive disease, where dementia symptoms gradually worsen over a number of years. In its early stages, memory loss is mild, but with late-stage AD, individuals lose the ability to carry on a conversation and respond to their environment. Those with AD live an average of eight years after their symptoms become noticeable to others, but survival can range up to 20 years, depending on age and other health conditions.

The most common early symptom of AD is difficulty remembering newly learned information because AD changes typically begin in the part of the brain that affects learning and memory. As AD advances through the brain it leads to increasingly severe symptoms, including disorientation, mood and behavior changes; deepening confusion about events, time and place; unfounded suspicions about family, friends and professional caregivers; more serious memory loss and behavior changes; and difficulty speaking, swallowing and walking.

The National Institute of Neurological and Communicative Disorders and Stroke (NINCDS) and the Alzheimer's Disease and Related Disorders Association (ADRDA, now known as the Alzheimer's Association) established the most commonly used NINCDS-ADRDA Alzheimer's Criteria for diagnosis in 1984, extensively updated in 2007. These criteria require that the presence of cognitive impairment, and a suspected dementia syndrome, be confirmed by neuropsychological testing for a clinical diagnosis of possible or probable AD. A histopathologic confirmation including a microscopic examination of brain tissue is required for a definitive diagnosis. Good statistical reliability and validity have been shown between the diagnostic criteria and definitive histopathological confirmation. Eight cognitive domains are most commonly impaired in AD: memory, language, perceptual skills, attention, constructive abilities, orientation, problem solving and functional abilities. These domains are equivalent to the NINCDS-ADRDA Alzheimer's Criteria as listed in the Diagnostic and Statistical Manual of Mental Disorders (DSM-IV-TR) published by the American Psychiatric Association. Beside symptomatic treatments to temporarily slow the worsening of dementia symptoms, AD has no current cure, and the current treatments cannot stop AD from progressing.

It should be appreciated that the methods of the invention, as well as the composition and kits described herein after, are suitable for treating and preventing any stage of AD, at any age and any conditions and symptoms associated therewith.

Since ApoE4 is involved in vascular pathologies, in yet some further embodiments, the methods of the invention may be applicable for any vascular pathology or condition. In some specific embodiments, such conditions may be at least one of cerebrovascular condition or disease and cardiovascular condition or disease.

In certain embodiments, the cerebrovascular condition or disease may comprise vascular, cognitive impairment disorders and conditions.

As indicated above, plaques and tangles are involved with AD as well as in other age-related neurodegenerative processes. Thus, it should be appreciated that the invention further encompasses the use of the ApoE4 targeted therapeutic methods and compositions disclosed herein for treating other age-related conditions, specifically cognitive decline. Thus, in some embodiments, the method of the invention may be applicable in treating and preventing Mild cognitive impairment (MCI), Age-related cognitive decline and Dementia with Lewy bodies (DLB).

The invention therefore in certain embodiments thereof, provides methods for treating, preventing, inhibiting, reducing, eliminating, protecting or delaying the onset of age-associated mild cognitive impairment (MCI).

“Age-associated mild cognitive impairment (MCI)”, as use herein is a condition that causes cognitive changes. MCI that primarily affects memory may be classified as “amnestic MCI” where the subjects experience impairment in memorizing information that relate to recent events, appointments or conversations or recent events. MCI that affects thinking skills other than memory is known as “nonamnestic MCI”. Thinking skills that may be affected by nonamnestic MCI include the ability to make sound decisions, judge the time or sequence of steps needed to complete a complex task, or visual perception.

Normal aging is associated with a decline in various memory abilities in many cognitive tasks; the phenomenon is known as age-related memory impairment (AMI), age-associated memory impairment (AAMI) or age-associated cognitive decline (ACD). The ability to encode new memories of events or facts and working memory shows decline in both cross-sectional and longitudinal studies. Studies comparing the effects of aging on episodic memory, semantic memory, short-term memory and priming revealed that episodic memory is especially impaired in normal aging; some types of short-term memory are also impaired. The deficits may be related to impairments seen in the ability to refresh recently processed information. Normally, there is little age-associated decline in some mental functions such as verbal ability, some numerical abilities and general knowledge but other mental capabilities decline from middle age onwards, or even earlier.

The latter include aspects of memory, executive functions, processing speed and reasoning. It should be therefore appreciated that in some embodiments, the invention provides methods and compositions for the treatment for any cognitive decline, specifically cognitive decline associated with age, specifically, the age of 50, 55, 60, 65, 70, 75, 80, 85, 90, 95 and more, years of age.

In yet some further specific embodiments, it should be appreciated that the method of the invention may be applicable for preventing and reducing age-related cognitive decline in apoE4 carriers who do not have an overt apparent disease but decline more than age matched subjects that are not carriers of apoE4. It should be noted that this may refer to normal aging which is accentuated in apoE4 carriers. It yet some further specific embodiments, the method of the invention may be suitable for treating and preventing cognitive decline in aging apoE4 carriers with sub clinical disease which given time will surface.

“Dementia with Lewy Bodies (DLB)”, as used herein, is a relatively common cause of dementia, estimated to account for up to 30% of dementia cases, and affecting up to 5% of those over the age of 75. Pathologically, it is defined by the presence of alpha synuclein containing Lewy bodies in the brain, but their distribution, affecting the neocortex, limbic system and brainstem. Clinically, DLB is characterized by a progressive dementia with prominent visual hallucinations and delusions, and parkinsonism with bradykinesia and rigidity but typically minimal tremor. Marked cognitive fluctuations are a common feature of this condition, with episodes of confusion, excessive somnolence, and incoherent speech which can revert to a near normal state within hours.

It should be appreciated that the method of the invention may be applicable for any stage, type, degree, phase, level of DLB, or for any symptom or condition associated therewith. In yet some more specific embodiments, the present invention provides a method for treating, preventing, reducing, attenuating, inhibiting and eliminating a disorder associated with MSA.

“Multiple system atrophy (MSA)”, is a neuropathology that includes cell loss and gliosis in nigrostriatal and olivopontocerebellar structures taking the form of glial cytoplasmic inclusions containing fibrillar alpha-synuclein within oligodendrocytes. It presents with autonomic dysfunction along with parkinsonism and cerebellar dysfunction in varying combinations, and is clinically classified as being either mainly cerebellar in its presentation (MSA-C) or mainly parkinsonian (MSA-P).

In yet some further embodiments, the methods of the invention may be applicable in treating, reducing and preventing any vascular, inflammatory or neurodegenerative condition that may involve at least one of accelerated age-related decrease in cortical thickness and hippocampal volume, Cerebral angiopathy, Impaired synaptic plasticity, synaptic loss, hippocampal atrophy, loss of dendritic spines, brain inflammation, neurovascular dysfunction, BBB-breakdown, leakage of blood derived toxic proteins into the brain and reduction in the length of small vessels, Frontotemporal dementia, Neuro-inflammation and impairment of neurogenesis.

More specifically, in some embodiments, the methods of the invention may be applicable for accelerated age-related decrease in cortical thickness and hippocampal volume. The term “accelerated age-related decrease in cortical thickness and hippocampal volume” refers to normal brain aging which is characterized by an overall cerebral atrophy. This atrophy is associated with shrinkage of grey matter (GM) and white matter (WM) volumes and enlargement of the cerebrospinal fluid (CSF) spaces.

In yet some further embodiments, the methods of the invention may be applicable for Cerebral amyloid angiopathy (CAA). The term CAA, also known as congophilic angiopathy, is a form of angiopathy in which amyloid deposits form in the walls of the blood vessels of the central nervous system. The term congophilic is used because the presence of the abnormal aggregations of amyloid can be demonstrated by microscopic examination of brain tissue by Congo red-staining. The beta amyloid material and deposits are found primarily in the brain.

Still further, in certain embodiments the methods of the invention may be applicable for treating Impaired synaptic plasticity. The term “Impaired synaptic plasticity” relates to the decrease or inability of a synapse to change its strength as a result of successive activations. Plastic change often results from the alteration of the number of neurotransmitter receptors located on a synapse. There are several underlying mechanisms that cooperate to achieve synaptic plasticity, including changes in the quantity of neurotransmitters released into a synapse and changes in how effectively cells respond to those neurotransmitters. Synaptic plasticity in both excitatory and inhibitory synapses has been found in some cases, to be dependent upon postsynaptic calcium release.

In yet some further embodiments, the methods of the invention may be applicable for synapse loss. The term “Synapse loss”, as used herein, refers to a loss of synaptic contacts in the brain of patients. It is associated with sensory, motor, and cognitive impairments in a variety of neurodegenerative conditions, such as major depressive disorder, schizophrenia, Alzheimer's disease, Huntington disease, and amyotrophic lateral sclerosis (ALS), as well as aging. Loss of excitatory synapses is the strongest correlate for cognitive impairments in Alzheimer's disease.

In some further embodiments, the methods of the invention may be applicable for hippocampal atrophy. The term “hippocampal atrophy” relates to a condition characterized by degeneration of the hippocampus. It is one of the characteristic features of hippocampal sclerosis and Alzheimer's disease. Hippocampal atrophy causes memory deprivation and spatial disorientation as well as difficulty in identifying smell.

In further embodiments, the methods of the invention may be applicable for loss of dendritic spines. The term “loss of dendritic spines” refers to a decrease in dendritic spine density in the brain of patients. Dendritic spines are small, dynamic protuberances from the dendritic shaft that are critical for synaptic transmission throughout the CNS, representing the primary location of excitatory glutamatergic neurotransmission Spine plasticity has been noted as a possible mechanism of long-term potentiation/depression and has been implicated in several models of learning and memory. Profound decreases in dendritic spine density, as well as alterations in spine shape and size, have been detected in both status epilepsy and several models of chronic epilepsy.

In certain embodiments, the methods of the invention may be applicable for brain inflammation. The term “brain inflammation” or Encephalitis relates to an inflammation of the brain tissue most commonly caused by viral infections but in rare cases may be caused by bacteria or even fungi. There are two main types of encephalitis: primary and secondary. Primary encephalitis occurs when a virus directly infects the brain and spinal cord. Secondary encephalitis occurs when an infection starts elsewhere in another part of the body and then raise the brain.

In other embodiments, the methods of the invention may be applicable for neurovascular dysfunction. The term “neurovascular dysfunction” relates to a damage in the neurovascular unit which comprises the interactions among glial, neuronal and vascular elements. Homeostatic signaling within the neurovascular unit is critical to normal brain function. The hemodynamic communication between neurons and the cerebrovasculature is necessary to efficiently couple Cerebral Blood Flow (CBF) to neuronal activation. Dysfunctional cell-cell signaling in the neurovascular unit is increasingly implicated as characteristic feature of CNS diseases. Structural and functional integrity of the CNS depends on the coordinated activity of the neurovascular unit to not only couple neural activity to CBF but also to regulate transport across the blood-brain barrier. There is some evidence that disturbance of the functional relationships among the cells of the neurovascular unit is an early event in Alzheimer's disease.

In some further embodiments, the methods of the invention may be applicable for Blood-brain barrier breakdown and any associated disorders. As used herein, the term “Blood-brain barrier breakdown” is the hallmark features of several brain pathologies and injuries. The BBB is mainly composed of the cerebral endothelial cells and the tight junctions (TJs) between them. TJs between the neighboring endothelial cells include transmembrane TJs, i.e. occludin, claudins, junctional adhesion molecules, etc., and membrane-bound TJs, i.e. zonula occludens. Zonula occludens play an important role in regulating BBB permeability by binding to both transmembrane tight junctions and actin cytoskeleton intracellularly. Various mediators of inflammation are shown to modulate BBB breakdown and permeability in a variety of pathologies. Blood-brain barrier breakdown and the associated hyperpermeability is the leading cause of brain edema and elevated intracranial pressure followed by decreased perfusion pressure leading to poor clinical outcomes in traumatic brain injury (TBI).

In some further embodiments, the methods of the invention may be applicable for leakage of blood derived toxic proteins into the brain and related disorders. As used herein, the term “leakage of blood derived toxic proteins into the brain” refers to the accumulation of blood-derived neurotoxic proteins in the CNS including fibrin, thrombin, hemoglobin, iron-containing hemosiderin, free iron and/or plasmin (an extracellular matrix-degrading enzyme) causing progressive neurodegeneration with loss of neurons mediated by either direct neuronal toxicity, oxidant stress and/or detachment of neurons from their supporting extracellular matrix.

Still further, in some embodiments, the methods of the invention may be applicable for conditions associated with reduction in the length of small vessels. The term “reduction in the length of small vessels” relates to Cerebral small vessel disease (SVD) denoting a range of pathological processes, which affect the small arteries, arterioles, capillaries and small veins of the brain. SVD is associated with small subcortical infarcts, lacunes, white matter hyperintensities, enlarged perivascular spaces, microbleeds, and cortical atrophy, involves in strokes and constitutes a major cause of cognitive decline, particularly in the elderly.

In some further embodiments, the methods of the invention may be applicable for Frontotemporal dementia. As used herein, the term “Frontotemporal dementia” (FTD) refers to a clinical presentation of frontotemporal lobar degeneration, which is characterized by progressive neuronal loss predominantly involving the frontal or temporal lobes, and typical loss of over 70% of spindle neurons, while other neuron types remain intact. Common signs and symptoms include significant changes in social and personal behavior, apathy, blunting of emotions, and deficits in both expressive and receptive language.

In yet some further embodiments, the methods of the invention may be applicable for conditions associated with neuroinflammation. The term “neuroinflammation” relates to an inflammation of the nervous tissue. It may be initiated in response to a variety of cues, including infection, traumatic brain injury, toxic metabolites, or autoimmunity. In the central nervous system (CNS), including the brain and spinal cord, microglia are the resident innate immune cells that are activated in response to these cues. The CNS is typically an immunologically privileged site because peripheral immune cells are generally blocked by the blood-brain barrier (BBB), a specialized structure composed of astrocytes and endothelial cells. However, circulating peripheral immune cells may surpass a compromised BBB and encounter neurons and glial cells expressing major histocompatibility complex molecules, perpetuating the immune response. Although the response is initiated to protect the central nervous system from the infectious agent, the effect may be toxic and widespread inflammation as well as further migration of leukocytes through the blood-brain barrier.

In certain further embodiments, the methods of the invention may be applicable for conditions associated with impairment of neurogenesis. The term “impairment of neurogenesis” refers to the Reduced level of production of neurons and is associated with cognitive functional impairments. Neurogenesis impairment is also an early event of Down syndrome and Alzheimer's disease.

Still further, in some embodiments, the method of the invention may be applicable in ApoE4 carriers that display a poor outcome following traumatic brain injury (TBI). In yet some further embodiments, the method of the invention improve the outcome following TBI of ApoE4 carriers, specifically when compared to non-treated carrier. More specifically, it should be understood that the method of the invention may be used for improving the recovery of ApoE4 carriers from TBI and any associated conditions.

An acute brain injury or traumatic brain injury (TBI) is an insult to the brain, caused usually by an external mechanical force, possibly leading to permanent or temporary impairment of cognitive, physical, and psychosocial functions, with an associated diminished or altered state of consciousness. The definition of TBI has not been consistent and tends to vary according to specialties and circumstances. Often, the term brain injury is used synonymously with head injury, which may not be associated with neurologic deficits. The definition also has been problematic with variations in inclusion criteria. TBI can be classified based on severity, mechanism (closed or penetrating head injury), or other features (e.g. occurring in a specific location or over a widespread area). Head injury usually refers to TBI, but is a broader category because it can involve damage to structures other than the brain, such as the scalp and skull. Brain trauma can be caused by a direct impact or by acceleration alone. In addition to the damage caused at the moment of injury, brain trauma causes secondary injury, a variety of events that take place in the minutes and days following the injury. These processes, which include alterations in cerebral blood flow and the pressure within the skull, contribute substantially to the damage from the initial injury. TBI can cause a host of physical, cognitive, emotional, and behavioral effects, and outcome can range from complete recovery to permanent disability or death.

TBI, as used herein includes those brain injuries occurring in motor vehicle accidents, after falls, caused by assault and in sports when force is applied to the head sufficiently to produce injury to the structure of the brain. Such injury can include bruising, tearing and swelling of brain tissue. It can include intracranial bleeding, such as subdural, epidural, subarachnoid, intraparenchymal and intraventricular hemorrhage. Brain tissue can be injured such as due to shearing of axons, even when little to no bleeding occurs. Despite extensive research over many years at several large clinical trials, there are currently no effective treatments for TBI other than meticulous supportive care.

One definition of TBI is provided in the Individuals with Disabilities Education Act which defines traumatic brain injury as “an acquired injury to the brain caused by an external physical force, resulting in total or partial functional disability or psychosocial impairment, or both, that adversely affects educational performance. The term as used herein applies to both open and closed head injuries resulting in impairments in one or more areas, such as cognition, language, memory, attention, reasoning, abstract thinking, judgment, problem-solving, sensory, perceptual, and motor abilities, psycho-social behavior, physical functions, information processing, and speech. By specifically targeting the ApoE4 protein, the methods and compositions of the invention provide an effective tool in improving at least one of the impairments involved in TBI, as described herein.

TBI occurs in people of all ages, including infants and children, young adults, adults and elderly. A similar definition applies to people of all ages, with the modification that work-related, cognitive, behavioral, emotional and social performance impairments can be involved in addition to adverse effects on educational performance.

In yet another embodiment, the invention may be applicable for treating chronic brain injuries. Chronic brain injuries are defined as conditions characterized by persistent brain damage or dysfunction as sequelae of cranial trauma. This disorder may result from diffuse axonal injury; intracranial hemorrhages; brain edema; recurrent brain injuries and other conditions. Clinical features may include dementia; focal neurologic deficits; persistent vegetative state; akinetic mutism; or coma. Chronic brain injury is sometimes referred to as post-traumatic, chronic encephalopathy, post-concussive chronic encephalopathy, chronic traumatic encephalopathy, chronic post-traumatic encephalopathy, chronic post-concussive syndrome, chronic post-concussive encephalopathy, brain, chronic injury and post-concussive syndrome.

In yet some further embodiments, as the method of the invention may be applicable in the treatment and prevention of dyslipidemia and hyperlipoprotienemia.

Still further, in some embodiments, the methods of the invention may be applicable for ischemic conditions. The term “ischemia” as herein defined refers to a restriction in blood supply to tissues, causing a shortage of oxygen and glucose needed for cellular metabolism. Ischemia is generally caused by blood vessels problems with resultant damage to or dysfunction of tissue. In some embodiments the ischemic condition is an Ischemic heart disease, e.g. Acute coronary syndrome, Angina pectoris, Angor animi, Coronary artery disease, Coronary ischemia, Hibernating myocardium, Mildronate, Myocardial infarction and Prinzmetal's angina.

In other embodiments the ischemic condition is ischemic stroke. A stroke (also referred to as cerebrovascular accident, CVA), is the rapid loss of brain function due to disturbance in blood supply to the brain. Stroke may be the result of ischemia (lack of blood flow) caused by blockage (which may be the result of thrombosis or arterial embolism). As a consequence, the affected area of the brain cannot function, which might result in an inability to move one or more limbs on one side of the body, among other symptoms. Thus, the term “ischemic stroke” as herein defined refers to an obstruction within a blood vessel supplying blood to the brain. There are various classification systems for acute ischemic stroke, some of them rely primarily on the initial symptoms. Based on the extent of the symptoms, the stroke episode may be classified as total anterior circulation infarct (TACI), partial anterior circulation infarct (PACI), lacunar infarct (LACI) or posterior circulation infarct (POCI). These four entities predict the extent of the stroke, namely the area of the brain affected, the ischemic brain damage involved, infarct volume, the neurologic deficit.

As discussed above, the ApoE4 variant is associated with Diabetes related pathologies. Moreover, Type 2 diabetes is a risk factor for AD and this effect is particularly pronounced in ApoE4 carriers. Thus, in yet some further embodiments, the methods, compositions and kits of the invention may be also applicable in treating and preventing diabetes. More specifically, diabetes related pathology includes decrease levels of brain insulin receptors and impaired brain insulin metabolism. Diabetes is mostly characterized by hyperglycaemia while hypoglycemia is the most prevalent clinical complication in the daily management of insulin-treated people with diabetes.

Hypoglycemia continues to be the limiting factor in the glycemic management of diabetes. Insulin-induced severe hypoglycemia is known to cause brain damage.

On the other end, the presence of diabetes and associated hyperglycemia has been shown to worsen the extent of neuronal damage following other forms of central nervous system insults (i.e., stroke) in both preclinical and clinical settings.

Likewise, type 2 diabetes is a risk factor for AD and this effect is particularly pronounced in ApoE4 carriers.

Therefore in some embodiments the methods and compositions of the invention are suitable for treating, preventing, ameliorating, inhibiting or delaying the onset of diabetes and diabetes related conditions. Specifically, diabetes type II, diabetes type I, gestational diabetes (occurs during pregnancy) or any diabetes related condition. In some embodiments all may be applicable in the present invention.

Diabetes mellitus is a syndrome characterized by disordered metabolism and inappropriately high blood sugar (hyperglycaemia) resulting from either low levels of the hormone insulin or from abnormal resistance to insulin's effects coupled with inadequate levels of insulin secretion to compensate. The characteristic symptoms are excessive urine production (polyuria), excessive thirst and increased fluid intake (polydipsia), and blurred vision, these symptoms are likely absent if the blood sugar is only mildly elevated.

Diabetes mellitus type II—formerly non-insulin-dependent diabetes mellitus (NIDDM) or adult-onset diabetes—is a metabolic disorder that is characterized by high blood glucose in the context of insulin resistance and relative insulin deficiency.

Insulin resistance means that body cells do not respond appropriately when insulin is present. Unlike type I diabetes mellitus, insulin resistance is generally “post-receptor”, meaning it is a problem with the cells that respond to insulin rather than a problem with the production of insulin. This is a more complex problem than type I, but is sometimes easier to treat, especially in the early years when insulin is often still being produced internally. Severe complications can result from improperly managed type II diabetes, including renal failure, erectile dysfunction, blindness, slow healing wounds (including surgical incisions), cataract, and arterial disease, including coronary artery disease. The onset of type II has been most common in middle age and later life, although it is being more frequently seen in adolescents and young adults due to an increase in child obesity and inactivity.

Diabetes is often initially managed by increasing exercise and dietary modification. As the condition progresses, medications may be needed. Unlike type I diabetes, there is very little tendency toward ketoacidosis though it is not unknown. One effect that can occur is nonketonic hyperglycemia. Long term complications from high blood sugar include an increased risk of heart attacks, strokes, amputation, and kidney failure.

There are many factors which can potentially give rise to or exacerbate type II diabetes. These include obesity, hypertension, elevated cholesterol (combined hyperlipidemia), and with the condition often termed metabolic syndrome (it is also known as Syndrome X, Reavan's syndrome, or CHAOS). Other causes include acromegaly, Cushing's syndrome, thyrotoxicosis, pheochromocytoma, chronic pancreatitis, cancer and drugs. Additional factors found to increase the risk of type II diabetes include aging, high-fat diets and a less active lifestyle. There is also a strong inheritable genetic connection in type II diabetes.

In some specific embodiments, the methods of the invention may be applicable specifically for mammalian subject that are carries at least one ApoEε4 allele.

As discussed above, such subjects may include heterozygotes that carry the ApoEε4 allele and the ApoEε3 allele or heterozygotes that carry the ApoEε2 allele and the ApoEε4 allele (ε3/ε4 or ε2/ε4, respectively), as well as homozygotes that carry the ApoEε4 at both alleles (ε4/ε4).

Still further, the cas protein used by the methods of the invention may be a member of at least one of CRISPR-associated system of Class 2 and class 1, specifically, any one of type II, type I, type III, type IV, type V, type VI.

In more specific embodiments, such cas protein may be a member of a CRISPR-associated system type II.

In some embodiments, the cas protein used by the method of the invention may be cas9 variant that specifically recognizes the 5′-NGCG-3′ PAM, or any derivative or fusion protein thereof.

In some specific embodiments, the cas9 variant used by the method of the invention may comprise at least one of D1135V, G1218R, R1335G and T1337R substitutions.

In more specific embodiments, the cas9 variant useful in the methods of the invention may be the SpCas9 VRER variant or any derivative or fusion protein thereof.

In certain embodiments, the SpCas9 VRER variant used by the methods of the invention may comprise the amino acid sequence as denoted by SEQ ID NO. 6.

In yet some further embodiments, the gRNA used by the methods of the invention may be a gRNA that target a protospacer comprising the nucleic acid sequence as denoted by SEQ ID NO. 18 of the ApoEε4 allele, or any fragments thereof.

In more specific embodiments, such gRNA may comprise the nucleic acid sequence as denoted by SEQ ID NO. 8. In some alternative embodiments, the methods and compositions of the invention may use gRNA comprising the nucleic acid sequence as denoted by any one of SEQ ID NO. 19, 20, 21 or 22, or any combinations thereof.

It should be understood that in some embodiments, where the ApoE rs28931579 is targeted, the gRNA used by the methods of the invention may be a gRNA that target a protospacer comprising the nucleic acid sequence as denoted by SEQ ID NO. 31 of the ApoE rs28931579 allele (ApoE4+), or any fragments thereof.

As discussed above, the method of the invention provide therapeutic methods for treating and preventing disorders or conditions associated with ApoE4.

As used herein, “disease”, “disorder”, “condition” and the like, as they relate to a subject's health, are used interchangeably and have meanings ascribed to each and all of such terms.

It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder, condition or pathology causes a second disease, disorder, condition or pathology.

As noted above, the invention provides methods for treating disorders as specified above. The term “treatment” as used herein refers to the administering of a therapeutic amount of the composition of the present invention, specifically, the CRISPR system discussed above, which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above. The treatment may be undertaken when a neuro-pathological, vascular or inflammatory condition initially develops, or may be a continuous administration, for example by administration more than once per day, every 1 day to 7 days, every 7 day to 15 days, every 15 day to 30 days, every month to two months, every two months to 6 months, or even more, to achieve the above-listed therapeutic effects.

As noted above, the invention further provides a prophylactic tool for preventing ApoE4 associated disorders, based on the existence of at least one ApoE4 allele in a subject, even before the appearance of any symptoms of the disease. The term “prophylaxis” refers to prevention or reduction the risk of occurrence of the biological or medical event, specifically, the occurrence or re-occurrence of disorders associated with neurodegeneration, inflammation and vascular pathologies, that is sought to be prevented in a tissue, a system, an animal or a human being, by a researcher, veterinarian, medical doctor or other clinician, and the term “prophylactically effective amount” is intended to mean that amount of an active ingredient administered will achieve this goal. Thus, in particular embodiments, the methods of the invention are particularly effective in the prophylaxis, i.e., prevention of conditions associated with any of the neurodegenerative, inflammatory or vascular disorders discussed herein. Thus, subjects treated by the methods of the invention or administered with the compositions are less likely to experience symptoms associated with said neurodegenerative, vascular and/or inflammatory disorders that are also less likely to re-occur in a subject who has already experienced them in the past.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the neurodegenerative, vascular and/or inflammatory disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with neurodegenerative, vascular and/or inflammatory disorders and their symptoms slowing their progress, further exacerbation or development, so as to appear later than in the absence of the treatment according to the invention.

As noted above, treatment or prevention include the prevention or postponement of development of the disease, prevention or postponement of development of symptoms and/or a reduction in the severity of such symptoms that will or are expected to develop. These further include ameliorating existing symptoms, preventing—additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process, specifically, any of the neurodegenerative, vascular and/or inflammatory disorder by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

The present invention relates to the treatment of subjects, or patients, in need thereof. By “patient” or “subject in need” it is meant any organism who may be affected by the above-mentioned conditions, and to whom the preventive and prophylactic compositions and methods herein described is desired. More specifically, the composition/s, kit/s and method/s of the invention are intended for preventing pathologic condition in mammals. By “mammalian subject” is meant any mammal for which the proposed therapy is desired, including humans, domestic and non-domestic mammals such as canine and feline subjects, bovine, simian, equine and murine subjects and rodents. It should be noted that specifically in cases of non-human subjects, the method of the invention may be performed using administration via injection, drinking water, feed, spraying, oral lavage and directly into the digestive tract of subjects in need thereof.

It should be appreciated that any systemic or local administration mode may be applicable in the present invention. Routes of administration of the ApoE4 targeting CRISPR system of the invention or any compositions thereof include, but are not limited to, intracerebral, intraventricular, intracerebroventricular, intrathecal, intracisternal, intraspinal and/or peri-spinal routes of administration by delivery via intracranial or intravertebral needles and/or catheters with or without pump devices. It should be appreciated that in some embodiments any further administration modes may be applicable, for example, intraperitoneal (IP), intravenous (IV) and intradermal, subcutaneous, nasal, oral and intramuscular, administration.

In some embodiments, for neuronal application specific procedures may be used in the present invention for applying the ApoE4 targeting CRISPR system of the invention or any compositions thereof at the specific neuronal tissue. For example, Stereotactic surgery or stereotaxy is a minimally invasive form of surgical intervention which makes use of a three-dimensional coordinate system to locate small targets inside the body and to perform on them some action such as ablation, biopsy, lesion, injection, stimulation, implantation, radiosurgery (SRS), traditionally and limited to brain surgery. In some embodiments, the ApoE4 targeting CRISPR system of the invention or any compositions thereof may be administered by the methods of the invention using Stereotactic surgery or stereotaxy.

In some specific embodiments, the administration may be targeted toward particular brain regions, more specifically, the hippocampus or the Entorhinal cortex.

The hippocampus, as used herein, is a major component of the brains of humans and other vertebrates. Humans and other mammals have two hippocampi, one in each side of the brain. The hippocampus belongs to the limbic system and plays important roles in the consolidation of information from short-term memory to long-term memory, and in spatial memory that enables navigation. The hippocampus is located under the cerebral cortex (allocortical) and in primates in the medial temporal lobe. It contains two main interlocking parts: the hippocampus proper and the dentate gyrus.

The entorhinal cortex (EC) (ento=interior, rhino=nose, entorhinal=interior to the rhinal sulcus) is an area of the brain located in the medial temporal lobe and functioning as a hub in a widespread network for memory and navigation. The EC is the main interface between the hippocampus and neocortex.

In yet a further aspect, the invention relates to a pharmaceutical composition comprising a therapeutic effective amount of: (a) at least one polypeptide comprising at least one cas protein, or any nucleic acid encoding said polypeptide, wherein said Cas protein specifically recognizes the 5′-NGCG-3′ PAM; and (b) at least one nucleic acid sequence comprising at least one gRNA that targets a protospacer located upstream to said PAM within at least one pathogenic allele of ApoE, for example, the ApoEε4 allele or the ApoEε4+ allele, or any nucleic acid sequence encoding said gRNA. In some alternative embodiments, the composition of the invention may comprise a construct, vehicle, vector, kit comprising (a) and (b). The compositions of the invention comprise two elements, specifically, at least one gRNA, and at least one Cas9 variant that recognize the PAM of the invention. It should be appreciated that both elements may be provided either as an gRNA and a polypeptide (cas9), or as nucleic acid sequences encoding these elements. In some embodiments, the nucleic acid sequence encoding the gRNA may be provided either alone or in a nucleic acid molecule that comprises also the nucleic acid sequence encoding the Cas9 polypeptide, specifically, in a single nucleic acid molecule or vector. In yet some further embodiments, the composition of the invention may optionally further comprise at least one of pharmaceutically acceptable carrier/s, diluent/s and/or excipient/s.

In some specific embodiments, the Cas protein comprised within the composition of the invention may be a member of at least one of CRISPR-associated system of Class 1 and Class 2, specifically, any one of type II, type I, type III, type IV, type V and type VI. In more specific embodiments, such cas protein may be a member of a CRISPR-associated system type II.

In more specific embodiments, the cas protein used in the composition of the invention may be CRISPR associated Cas9 variant that specifically recognizes the 5′-NGCG-3′ PAM, or any derivative or fusion protein thereof.

In more specific and non-limiting embodiments, such Cas9 variant may comprise at least one of D1135V, G1218R, R1335G and T1337R substitutions. It should be understood that the particular positions refer to these specific positions within the Cas9 amino acid sequence as denoted by SEQ ID NO. 28.

In further embodiments, the Cas9 variant comprised within the composition of the invention may be the SpCas9 VRER variant or any derivative or fusion protein thereof. In more specific embodiments, such SpCas9 VRER variant may comprise the amino acid sequence as denoted by SEQ ID NO. 6.

In yet some further embodiments, the composition of the invention may comprise at least one gRNA that targets a protospacer comprising the nucleic acid sequence as denoted by SEQ ID NO. 18 of the ApoEε4 allele, or any fragments thereof.

In yet some particular embodiments, gRNA useful in the compositions of the invention may comprise the nucleic acid sequence as denoted by SEQ ID NO. 8. In some alternative embodiments, the methods and compositions of the invention may use gRNA comprising the nucleic acid sequence as denoted by any one of SEQ ID NO. 19, 20, 21 or 22, or any combinations thereof.

In yet some alternative embodiments, where the ApoE rs28931579 allele is targeted, the methods and compositions of the invention may use gRNA comprising the nucleic acid sequence as denoted by SEQ ID NO. 31.

In certain embodiments, the composition of the invention may be applicable for use in a method for treating, preventing, ameliorating, inhibiting or delaying the onset of an ApoE4 associated pathologic condition or disease in a mammalian subject.

In some specific embodiments, such ApoE4 associated pathologic condition or disease may be at least one of an acute or chronic vascular, inflammatory and neurodegenerative pathology or condition.

In some specific embodiments, the composition of the invention may be used for neurodegenerative disorder that may also involve vascular and/or inflammatory causes. In certain embodiments, such disorder may be Alzheimer's disease.

In some further embodiments, the composition of the invention may be used for vascular pathology or condition, more specifically, at least one of cerebrovascular condition or disease and cardiovascular condition or disease.

In yet some further embodiments, the composition of the invention may be used in cerebrovascular condition or disease. More specifically, vascular cognitive impairment disorders and conditions.

In more particular embodiments, the composition of the invention may be applicable in treating vascular cognitive impairment including stroke vascular dementia, MCI, Age-related cognitive decline and DLB.

Still further, in some embodiments, the compositions provided by the invention may be applicable in any vascular, inflammatory or neurodegenerative condition that may comprise at least one of accelerated age-related decrease in cortical thickness and hippocampal volume, Cerebral angiopathy, Impaired synaptic plasticity, synaptic loss and impairments, hippocampal atrophy, loss of dendritic spines, brain inflammation, neurovascular dysfunction, BBB-breakdown, leakage of blood derived toxic proteins into the brain and reduction in the length of small vessels, Frontotemporal dementia, Neuro-inflammation and impairment of neurogenesis.

Still further, in some embodiments, the compositions of the invention may be applicable in treating and improving outcome following TBI.

In yet some specific embodiments, the compositions of the invention may be applicable for any mammalian subject, that carries at least one ApoEε4 allele, specifically, any carrier of at least one ApoEε4 allele that suffers from any of the ApoE4 disorder disclosed herein before. It should be appreciated that the invention further provides methods, compositions and kits that may be applicable for any disorder or condition associated with the ApoE4+ allele. In some embodiments, such disorders are any of the disorders disclosed by the invention.

It should be appreciated that the pharmaceutical composition of the invention may comprise the active compound, specifically, the ApoE4 targeting CRISPR system provided by the invention, in free form and be administered directly to the subject to be treated. Alternatively, admixing it in a pharmaceutically acceptable carrier prior to administration may be desirable. Therapeutic formulations may be administered in any conventional dosage formulation. Formulations typically comprise at least one active ingredient, as defined above, together with one or more pharmaceutically and physiologically acceptable carriers in the sense of being compatible with the other ingredients and not injurious to the patient.

In some specific embodiments, the pharmaceutical composition of the invention may be suitable for injection. The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

The pharmaceutical compositions of the invention generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

The pharmaceutical compositions of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome containing formulations.

Formulations include those suitable for intracerebral, intraventricular, intracerebroventricular, intrathecal, intracisternal, intraspinal and/or peri-spinatopical, oral, nasal, rectal, nasal, or parenteral (including subcutaneous, intramuscular, intraperitoneal (IP), intravenous (IV) and intradermal) administration. The nature, availability and sources, and the administration of all such compounds including the effective amounts necessary to produce desirable effects in a subject are well known in the art. The preparation of pharmaceutical compositions is well known to the skilled man of the art.

In yet some further aspect thereof, the invention provides the use of a therapeutic effective amount of (a) at least one polypeptide comprising at least one Cas protein, or any nucleic acid encoding said polypeptide, more particularly, the cas protein specifically recognizes the 5′-NGCG-3′ PAM; and (b) at least one nucleic acid sequence comprising at least one gRNA that targets a protospacer located upstream to said PAM within the ApoEε4 allele, and/or at least one nucleic acid sequence comprising at least one gRNA that targets a protospacer located upstream to said PAM within the ApoE rs28931579 (ApoE4+) or any nucleic acid sequence encoding said gRNAs, in the preparation of a composition for treating, preventing, ameliorating, inhibiting or delaying the onset of an ApoE4 associated pathologic condition or disease in a mammalian subject.

In some embodiments, the ApoE pathologic condition and specifically, the ApoE4 associated pathologic condition or disease may be at least one of an acute or chronic vascular, inflammatory and neurodegenerative pathology or condition.

In yet some further embodiments, the vascular pathology or condition may be at least one of cerebrovascular condition or disease and cardiovascular condition or disease.

In certain embodiments, cerebrovascular condition or disease may comprise vascular cognitive impairment disorders and conditions.

In yet some further embodiments, the invention provides the use of the compositions or the kits of the invention in treating a neurodegenerative disorder such as Alzheimer's disease.

In certain embodiments, the invention provides the use of the compositions and kits of the invention or any components thereof for treating and preventing vascular cognitive impairment, specifically, MCI, Age-related cognitive decline and DLB.

Still further, the invention provides the use of the compositions and kits of the invention or any components thereof in treating any cerebrovascular, inflammatory or neurodegenerative condition that may exhibit at least one of accelerated age-related decrease in cortical thickness and hippocampal volume, Cerebral angiopathy, Impaired synaptic plasticity, hippocampal atrophy, synaptic impairment and loss, loss of dendritic spines, brain inflammation, neurovascular dysfunction, BBB-breakdown, leakage of blood derived toxic proteins into the brain and reduction in the length of small vessels, Frontotemporal dementia, Neuro-inflammation and impairment of neurogenesis.

In some specific embodiments, the use provided by the invention may be applicable for improving recovery from TBI, specifically in ApoE4 carriers. Still further in some embodiments, the invention provides the use of any of the compositions or kits of the invention of any components thereof for any mammalian subject that carry at least one ApoEε4 allele. In some embodiments, subjects that carry at least one ApoEε4 allele, may be heterozygotes that carry the ApoEε4 allele and the ApoEε3 allele or heterozygotes that carry ApoEε2 allele and the ApoEε4 allele (ε3/ε4 or ε2/ε4, respectively), as well as homozygotes that carry the ApoEε4 at both alleles (ε4/ε4). It should be noted that, the minor allele in SNP rs429358 in some embodiment is C (cytosine), while the major allele in SNP rs429358 is T (thymine). In yet some further embodiments, the minor allele in SNP rs7412 is T, while the major allele of SNP rs7412 is C.

In certain embodiments, the cas protein provided by the use of the invention may be a member of at least one of CRISPR-associated system of Class 1 and Class 2, specifically, any one of type II, type I, type III, type IV, type V and type VI. More specifically, in some embodiments, such cas protein may be a member of a CRISPR-associated system type II. In some specific embodiments, the cas protein may be a cas9 variant that specifically recognizes the 5′-NGCG-3′ PAM, or any derivative or fusion protein thereof. In yet some further embodiments, the Cas9 variant comprises at least one of D1135V, G1218R, R1335G and T1337R substitutions.

In still further embodiments, the Cas9 variant may be the SpCas9 VRER variant or any derivative or fusion protein thereof. In yet some further embodiments, the SpCas9 VRER variant used by the invention may comprise the amino acid sequence as denoted by SEQ ID NO. 6.

In some embodiments, the gRNA used by the invention may target a protospacer comprising the nucleic acid sequence as denoted by SEQ ID NO. 18 of the ApoEε4 allele, or any fragments thereof.

Still further, such gRNA may comprise the nucleic acid sequence as denoted by SEQ ID NO. 8. In some alternative embodiments, the methods and compositions of the invention may use gRNA comprising the nucleic acid sequence as denoted by any one of SEQ ID NO. 19, 20, 21 or 22, or any combinations thereof.

Still further, the invention provides in some aspects thereof an effective amount of (a) at least one polypeptide comprising at least one Cas protein, or any nucleic acid encoding said polypeptide that specifically recognizes the 5′-NGCG-3′ PAM; and (b) at least one nucleic acid sequence comprising at least one gRNA that targets a protospacer located upstream to said PAM within the ApoEε4 allele, and/or at least one nucleic acid sequence comprising at least one gRNA that targets a protospacer located upstream to said PAM within the ApoE rs28931579 (ApoE4+) or any nucleic acid sequence encoding said gRNAs, for use in targeted elimination of at least one pathogenic allele of ApoE, and/or for use in treating, preventing, ameliorating, inhibiting or delaying the onset of an ApoE4 associated pathologic condition or disease in a mammalian subject.

In yet a further aspect, the invention provides a diagnostic method for the detection of a pathogenic ApoE allele in a subject. In some specific embodiments, the methods of the invention may comprise the following steps:

In a first step (a), contacting at least one biological sample of the subject with an effective amount of: (i) at least one polypeptide comprising at least one nuclease-dead CRISPR-associated protein (dCas), or any nucleic acid encoding such dCas polypeptide. It should be noted that the dCas protein specifically recognizes the 5′-NGCG-3′ PAM. The sample is also contacted with (ii) at least one gRNA that targets a protospacer located upstream to said PAM within the ApoE pathogenic allele, or any nucleic acid sequence encoding said gRNA.

In some embodiments the at least one of the dCas of (i) and the gRNA of (ii) may be either directly or indirectly attached, connected, conjugated, associated or fused to at least one detectable moiety. It should be understood that such step of contacting the dCas and gRNA with the biologic sample may be performed to allow the formation of a dCas9/sgRNA complex in the sample.

The second step (b), involves determining if at least one detectable signal from the at least one detectable moiety is detected in the sample of (a). In some embodiments, the detection of such detectable signal indicates the presence of at least one pathogenic ApoE allele in the sample, specifically a biological sample that comprise genomic DNA, and thereby, in the tested subject.

In some specific embodiments, the ApoE pathogenic allele may be the ApoEε4 allele.

In yet some other alternative or additional embodiments, the ApoE pathogenic allele may be the rs28931579 allele (also referred to herein as ApoE4+).

In some embodiments, the dCas protein used by the methods of the invention may be a defective variant of CRISPR associated protein 9 (Cas9) that further comprises at least one of Valine at position 1135, Arginine at position 1218, Glutamine at position 1335 and Arginine at position 1337 or any derivative or fusion protein thereof.

As indicated above, the nucleases, and specifically, the guided nucleases such as Cas9 used by the methods and kits of the invention may be in some embodiments, catalytically inactive nucleases. In such cases, only the targeting properties of these guided nucleases are used (e.g., targeting a target nucleic acid sequence using gRNA), for targeted binding and thereby detection of a target sequence, specifically, a pathogenic allele of ApoE. The nucleolytic activity in such cases is undesired. In some embodiments, a guided nuclease with no nucleolytic activity may be used. Thus, in some particular and non-limiting embodiments, the Cas9 enzyme used for the methods and kits of the invention may be a Cas9 devoid of any nucleolytic activity, for example, a defective enzyme such as dCas9. dCas9 is a mutant Cas9 that lacks endonucleolytic activity. A non-limiting example for such mutant may be a mutant that carries a point mutation in at least one of D10A (aspartic acid to alanine in position 10) and H840A (histidine to alanine in position 840). It should be noted that the specified positions relate to the corresponding positions in the wild type Cas9 protein, specifically, the spCas that may in some embodiments, comprise the amino acid sequence as specifically denoted by SEQ ID NO. 28.

In some embodiments, the detectable moiety may be connected directly to the dCas used by the method of the invention. More specifically, in such embodiments, the dCas may be provided as a fusion protein with such detectable moiety. In some specific and non-limiting embodiments, the dCas used by the diagnostic method of the invention may be provided as a fusion protein with a detectable moiety such as green flurorecent protein (GFP), red flurorecent protein (RFP), blue flurorecent protein (BFP) and the like, specifically, dCas-GFP. In such case, the sample, that comprises genomic DNA of the tested subject may be contacted with a dCas-GFP that specifically recognizes the 5′-NGCG-3′ PAM sequence of the invention. In case the specific pathogenic ApoE allele is present in the sample, specifically, the genomic DNA of the tested subject, the gRNA provided by the invention that targets a protospacer located upstream to said PAM within the ApoE pathogenic allele, will direct the dCas-GFP to specifically bind said PAM in the ApoE pathogenic allele, and a detectable signal will be detected. Detection of a detectable signal reflects and thereby indicates the presence of the ApoE pathogenic allele in the sample. In case no such PAM is present in the specific location of the ApoE gene, no binding occurs and no signal is detected.

As indicated herein above, the methods and kits of the invention may use a fusion protein, e.g., dCas-fused to a detectable moiety, for example, dCas-GFP. Fusion protein as used herein relates to a polypeptide composed of at least two different polypeptides prepared recombinantly or synthetically. In some embodiments, the fusion protein may comprise at least one linker connecting between the proteins or polypeptides.

In yet some alternative embodiments, the detectable moiety may be connected indirectly to one of the elements used by the methods of the invention, specifically, the gRNA provided by the invention, that targets a protospacer located upstream to said PAM within the ApoE pathogenic allele. Thus, in some particular embodiments, the gRNA provided by the methods of the invention may further comprise at least one or a plurality of detectable moiety binding sites, for example, fluorescent label binding sites. In yet some further specific embodiments, the gRNA of the invention may further comprise as the detectable moiety binding sites, at least one stem-loop sequence. In some specific embodiments, such stem and loop sequences that act as binding sites for the detectable moiety may include, but is not limited to, an MS2 stem loop sequence, a PP7 stem loop sequence or a BoxB stem loop sequence. In some specific embodiments, a gRNA that comprises at least one of an MS2 stem loop sequence, a PP7 stem loop sequence and a BoxB stem loop sequence may be used. Thus, in case the specific pathogenic ApoE allele is present in the sample, specifically, in the genomic DNA of the tested subject, the gRNA provided by the invention, that targets a protospacer located upstream to said PAM within the ApoE pathogenic allele will direct the dCas to specifically bind said PAM in the ApoE pathogenic allele. Following binding of this complex to the genomic DNA, a fusion protein comprising MS2-binding protein (MBP) fused to a detectable moiety (or alternatively, any fusion protein that comprises a detectable label and a protein that binds the stem and loop sequences comprised within the gRNA used by the invention), for example fluorophore (e.g. GFP) that has been added to the sample, binds the MS2 domain. Thus, the detectable signal is bound to the complex that recognizes the target ApoE pathogenic allele. Detection of a detectable signal indicates therefore the presence of the ApoE pathogenic allele in the sample. In case no PAM is present in the specific location of the ApoE gene, no binding occurs and no signal is detected. In some specific embodiments, the method of the invention may be directed at detecting the presence of the ApoEε4 allele in a subject, thereby providing diagnosis and prognosis of at least one disorder associated with the presence of said allele. In such case, the method of the invention may comprise in some embodiments thereof, contacting a sample of a subject with at least one dCas that recognizes the 5′-NGCG-3′ PAM, and at least one gRNA that comprises at least one of an MS2 stem loop sequence, a PP7 stem loop sequence and a BoxB stem loop sequence, for example, MS2 stem loop sequence. It should be noted that this gRNA specifically recognizes and binds a protospacer within the ApoEε4 allele, that is located upstream to the 5′-TGCG-3′ PAM. This particular SNP distinguishes between the pathogenic ApoEε4 and the ApoEε3 allele. Upon addition of MBP fused to a detectable moiety, for example, MBP-GFP, binding to the gRNA-MS2 stem loop sequence occurs. Such binding forms a complex of dCas-gRNA-MS2-MBP-GFP that is bound specifically to the genomic DNA in the tested sample, in the ApoEε4 specific loci. This complex is detectable due to the presence of the attached detectable moiety, .g., GFP. More specifically, in the presence of the ApoEε4 allele in the genomic DNA of the sample, the gRNA-MS2 stem loop sequence with the MBP-GFP complex connected to it, targets the dCas to the ApoEε4 specific PAM and a detectable signal may be detected, thereby indicating the presence of the pathogenic allele in the examined subject. It should be appreciated that in some embodiments, this method may enable detection of more than one pathogenic allele of the ApoE gene, for example, the rs28931579 allele, using gRNAs associated with MS2 stem loop sequence that may recruit MBP-fused to different detectable moieties, for example, BMP-GFP that binds the gRNA that targets the ApoEε4 allele and BMP-RFP that binds gRNA that targets, the rs28931579 allele.

In more specific embodiments, the gRNA sequence comprises at plurality of hairpin turns (e.g., stem loops). In one embodiment, the stem loops include, but are not limited to MS2, PP7 and BoxB. Although it is not necessary to understand the mechanism of an invention, it is believed that these hairpin turns can establish a broad spectral range for multi-loci labeling. For example, a variety of combinations of these hairpin turns are contemplated such that each gRNA recruits a different pair of fluorescent proteins (FPs) recognizing two RNA elements.

As used herein the term MS2 and PP7 relates to RNA aptamers that recruit the corresponding MS2 coat proteins (MCP) or PP7 coat proteins (PCP). Similar to the MCP-MS2 and PCP-PP7 systems, there are other RNA binding protein-aptamer systems (e.g. Com-com and NN2-BoxB) that may be used in gRNA scaffold design.

As noted above, the detection of the pathogenic allele of ApoE, requires in some embodiments of the diagnostic methods and kits of the invention, the use of a detectable moiety that may be directly or indirectly attached to at least one of the elements used by the methods and kits of the invention. The term “detectable moiety” are used herein, to refer to any composition detectable by spectroscopic, photochemical, biochemical, immunochemical, electrical, optical or chemical means. Such moieties include fluorescent dyes (e.g., fluorescein, texas red, rhodamine, green fluorescent protein, and the like), biotin for staining with labeled streptavidin conjugate, magnetic beads (e.g., Dynabeads (R)), radiolabels (e.g., H, I, S, C, or P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. The moieties contemplated in the present invention may be detected by various methods. For example, fluorescent markers may be detected using a photodetector to detect emitted light, while radiolabels may be detected using photographic film or scintillation counters. Enzymatic labels are typically detected by providing the enzyme with a substrate and detecting the reaction product produced by the action of the enzyme on the substrate, and calorimetric labels are detected by simply visualizing the colored label.

As used herein, the method of the invention (as well as the kit of the invention) encompasses the use of a biological sample of a subject. It should be noted that certain embodiments of the invention contemplate the use of different biological samples for the diagnosis and prognosis methods of the invention. In some specific embodiments, the term “sample” in the present specification and claims is meant to include biological samples. Biological samples may be obtained from mammal, specifically, a human subject, include fluid, solid (e.g., stool) or tissues, including hair and nail samples. The term “sample” may also include body fluids such as whole blood sample, blood cells, bone marrow, lymph fluid, serum, plasma, urine, sputum, saliva, faeces, semen, spinal fluid or CSF, the external secretions of the skin, respiratory, intestinal, and genitourinary tracts, tears, milk, any human organ or tissue, any biopsy, for example, brain, lymph node or spleen biopsies, any sample taken from any tissue or tissue extract, any sample obtained by lavage optionally of the breast ductal system, plural effusion, samples of in vitro or ex vivo cell culture and cell culture constituents. Some samples that are a priori not liquid may be contacted with a liquid buffers which are then used according to the diagnostic methods and kits of the invention. Still further, as used herein, the term “sample” refers to cells, sub-cellular compartments thereof, tissue or organs. The tissue may be a whole tissue, or selected parts of a tissue. Tissue parts can be isolated by micro-dissection of a tissue, or by biopsy, or by enrichment of sub-cellular compartments. It should be further appreciated that the term sample as used herein further encompasses any preparation or extract prepared from any of the samples indicated above. In some non-limiting embodiments, genomic DNA prepared from any of the samples mentioned herein may be used as a biological sample in the methods and kits described herein. Biological samples may be obtained from all of the various families of domestic animals, as well as feral or wild animals, including, but not limited to, such animals as ungulates, bear, fish, lagamorphs, rodents, etc. Preferably, the sample is liquid, specifically, a body fluid sample, most preferably, a serum sample and of mammalian origin, specifically, human.

Still further, it should be understood that when using a nuclease-inactive version of Cas9, termed dCas9 (d for nuclease—dead), and by attaching either directly or indirectly a detectable moiety, specifically a fluorescent reporter to it or to the gRNA used by the kits and methods of the invention, it may be possible to deploy the CRISPR system as a probe to label specific genomic sequences, specifically, pathogenic alleles of ApoE, in living eukaryotic cells. In contrast to the technique of fluorescence in situ hybridization (FISH)—a classical method of considerable utility for many purposes, CRISPR-based labeling offers an advantage of allowing specific chromosomal loci to be spatially mapped in the live cell, and also is very straightforward to carry out as it involves simple DNA transfection of the cells.

It should be noted that the first step of the diagnostic methods of the invention comprises contacting a biological sample with the dCas and the gRNA. As used herein the term “contacting” refers to the positioning of the kit of the present invention or any component thereof, the polypeptide, specifically the dCas of the invention or any fusion protein thereof and/or gRNA or any nucleic acid sequence encoding the same, such that they are in direct or indirect contact with the sample or any nucleic acid sequence derived therefrom. Thus, the present invention contemplates both applying the polypeptide and/or gRNA of the present invention or a kit or composition thereof to a sample containing said DNA.

In yet a further aspect, the invention provides a diagnostic kit comprising: (a) at least one polypeptide comprising at least one dCas or any fusion protein thereof, or any nucleic acid sequence encoding the dCas polypeptide. It should be noted that the dCas protein specifically recognizes the 5′-NGCG-3′ PAM. The kit of the invention further comprises (b), at least one gRNA that targets a protospacer located upstream to the PAM within the ApoE pathogenic allele, or any nucleic acid sequence encoding this gRNA.

In some embodiments, at least one of the dCas of (a) and/or the gRNA of (b) may be either directly or indirectly attached, connected, conjugated, associated or fused to at least one detectable moiety.

In some specific embodiments, the ApoE pathogenic allele may be the ApoEε4 allele.

In yet some other alternative or additional embodiments, the ApoE pathogenic allele may be the rs28931579 allele.

In some embodiments, the kit of the invention may comprise a sheet of instructions for detecting the ApoE pathogenic allele; and optionally any reagents and/or buffer/s compatible with said dCas protein and said gRNA, and any reagent/s and/or buffers required to allow the formation of the gRNA/dCas complex. In some embodiments the kits of the invention may comprise any suitable means allowing the detection of the detectable signal formed by the detectable moiety attached directly or indirectly as specified herein to the gRNA/dCas complex in the sample.

In one embodiment, the dCas9 protein of the kit of the invention may be provided in some embodiments directly connected to at least one detectable moiety. In some specific embodiments, such dCas9 may be provided as a fusion protein with such detectable moiety, that may be in some embodiments a green fluorescent protein (GFP). It should be appreciated that any of the detectable moieties described herein before are also applicable for the kits of the invention.

In some embodiments, the kit of the invention may provide the detectable moiety indirectly connected to the components of the kit, specifically the gRNA. In these embodiments, the kit of the invention may comprise at least one gRNA (directed against a protospacer located upstream to the PAM of the invention within the ApoE pathogenic allele) that is indirectly connected to at least one or a plurality of fluorescent label binding sites.

In some embodiments, the plurality of detectable moiety binding sites, for example, fluorescent label binding sites bind a fluorescent protein.

In some embodiments, the detectable moiety binding sites may comprise at least one stem loop sequence included in the gRNA of the kit of the invention. In some embodiments, the at least one stem loop sequence may include, but is not limited to, an MS2 stem loop sequence, a PP7 stem loop sequence or a BoxB stem loop sequence. It should be appreciated that suitable stem loop sequences are those described in connection with the diagnostic methods of the invention. In certain embodiments, the gRNA sequence may comprise one fluorescent protein bound to one stem loop sequence. As noted above, the use of gRNA indirectly connected to a detectable moiety allows detection of at least one loci, and in some embodiments, multiple loci. Thus, in some embodiments, the gRNA sequence provide by the kit of the invention may comprise two fluorescent proteins, wherein each fluorescent protein is bound to a different stem loop sequence. In other embodiments, the gRNA sequence of the kit of the invention may comprise three fluorescent proteins, wherein each fluorescent protein is bound to a different stem loop sequence. It should be appreciated that the kits of the invention further contemplates the use of even 4, 5, 6, 7, 8, 9, 10 or more different detectable moieties, e.g., fluorescent proteins with different colors. In some embodiments, the at least one color includes, but is not limited to, red, green, blue, cyan, yellow, magenta or white. In some embodiments, the different color may be selected from the group consisting of red, green and blue.

In some embodiments, the diagnostic methods and kits of the invention may be used for detecting at least one gene target loci in a pathogenic allele of ApoE, for example, two gene target loci, three gene target loci, four gene target loci, five gene target loci or six gene target loci. In some embodiments, the identification of said at least one gene target loci is simultaneous. In some further embodiments, each of said at least one fluorescent protein has a different color.

In some embodiments, the kits of the invention may also optionally include appropriate systems (e.g. opaque containers) or stabilizers (e.g. antioxidants) to prevent degradation of the reagents by light or other adverse conditions.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

It should be appreciated that some embodiments consider the kit of the invention in compartmental form. More specifically, the different components of the kit of the invention may be provided in some embodiments in different containers, a plurality of vessels (test tubes), plates, micro-wells in a micro-plate, each containing one of the components provided, e.g., gRNA, dCas, detectable moieties, reagents and the like.

As indicated above, the invention further provides diagnostic methods and kits for identifying subjects that carry a pathogenic allele of the ApoE gene. However, it should be understood that by identifying carriers of pathogenic alleles, the invention further provides diagnosis of any diseases and conditions associated with the pathogenic allele detected. The term “diagnosis” refers to the process of determining which disease or condition explains a person's symptoms and signs. The information required for diagnosis is typically collected from a history and physical examination of the person seeking medical care. Often, one or more diagnostic procedures, such as diagnostic tests, are also done during the process. The method of the invention therefore provides a method for the diagnosis of any of the conditions and diseases associated with pathogenic allele of ApoE, specifically, the ApoE4 allele. More specifically, diagnosis of any of the disorders described by the invention. Still further, since some of the conditions associated with the existence of a pathogenic allele of ApoE are also associated with chances of a subject to recover from a disease, the diagnostic methods and kits of the invention further provides prognostic methods and kits. The term “prognosis” is defined as a forecast of the future course of a disease or disorder, based on medical knowledge. A complete prognosis includes the expected duration, function, and description of the course of the disease, such as progressive decline, intermittent crisis, or sudden, unpredictable crisis as well as the likelihood of a patient to survive.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols which are known in the art and not specifically described herein are generally followed as in Sambrook & Russell, 2001.

### Reagents

### Restriction Enzymes

BmgBI enzyme (NEB # R0628S)
Esp3I enzyme (ThermoFisher # FD0454)

### Plasmids

MSP1101 plasmid (comprising nucleic acid sequence as denoted by SEQ ID NO 7, Addgene, plasmid cat. No. #65773).
Lenti_sgRNA_EFS_GFP (LRG) plasmid (Addgene, plasmid cat. No. #65656).

### Kits

### QIAprep Midiprep (Qiagen Inc);

Quick ligase (BIOLINE Inc)

### Antibodies

anti-ApoE antibody (Mercury, catalogue number MBS 178479)
a donkey anti-goat secondary antibody (Jackson 705035147)

### Cells

Human ApoE3 or ApoE4 homozygous knock-in mouse astrocytic cells.

### Experimental Procedures

### Design and Construction of the CRISPR System

A variant of Cas9 was used named spCas9 VRER [12], [13] which differs from spCas9 in only 4 amino acids i.e. D1135V/G1218R/R1335E/T1337 (the VRER variant comprises amino acid sequence as denoted by SEQ ID NO 6, encoded by the nucleic acid sequence as denoted by SEQ ID NO. 5) and recognizes a unique sequence of PAM: NGCG. This specific spCas9 VRER variant was produced in bacteria using the MSP1101 plasmid (SEQ ID NO. 7), and verified by sequencing.

The sgRNA sequence was cloned into the Lenti_sgRNA_EFS_GFP (LRG) plasmid. The appropriate gRNA targeting the ApoEε4 allele was ordered as ssDNA oligos (IDT). The resulting PCR product was incorporated into LRG plasmid using Esp3I restriction site. More specifically, Oligo 1: 5′-CACCGGGCGCGGACATGGAGGACG-3′ also denoted by SEQ ID NO. 9 and Oligo 2: 3′-CCCGCGCCTGTACCTCCTGCCAAA-5′, also denoted by SEQ ID NO. 10, were used for an annealing reaction performed as follows: 1 μl of each oligo (100 μM), 1 μl 10× T4 ligation buffer, 0.5 μl T4 PNK in a total reaction volume of 10 μl.

The PCR program used included:

37° C.—30 min, 95° C.—5 min and then ramping down to 25° C. at 5° C. per min.

The annealing product was diluted to 1:200.

The ligation reaction was performed as follows: 50 ng digested plasmid (LRG), 1 μl diluted dsOligo duplex, 5 μl 2× Quick ligase Buffer, 1 μl Quick ligase (BIOLINE Inc). The ligation product (LRG:gRNA) was then transformed into bacterial competent cells JM109 (PROMEGA P9751). Plasmid purification was further performed using QIAprep Midiprep.

### Culture and Co-Transfection of Cells

The above-described CRISPR system was introduced into the Human ApoE3 or ApoE4 homozygous knock-in mouse astrocytic cells. Cells were grown in Dulbeco's Modified Eagle's Medium (DMEM) F12:HAM supplemented with gentamicin, sodium pyruvate and serum. Cells were incubated at 37° C. At the day before transfection, 200×10³ cells were seeded on a 6-well plate without antibiotics, thus reaching 70-90% of confluence. Lipofectamine 3000 reagent (Invitrogen) was used for transfecting both plasmids (MSP1101 and LRG) into the cells as follows: an amount of 1.25 μg from each plasmid (VRER Cas9 and the gRNA plasmids MSP1101 and LRG, respectively) was diluted in 125 μl Opti-MEM medium supplemented with 5 μl P3000 reagent and incubated for 15 minutes at RT. An amount of 250 μl of the DNA-Lipofectamine mix was added to the well and cells were incubated at 37° C.

The medium was changed on the following day, and cells were harvested and seeded on 10 cm plates. The medium of cells was collected 3 days after seeing for protein analysis whereas genomic DNA was produced on the day following co-transfection. DNA and medium samples were kept in −80° C.

### DNA Analysis

DNA was extracted from cells on the day following co-transfection and a PCR reaction was performed to amplify a 242 bp amplicon containing a part of ApoE gene using the following primers:

Forward:
SEQ ID NO. 11
5′-GGACGAGACCATGAAGGAGT-3′
Reverse:
SEQ ID NO. 12
5′-GCAGCTTGCGCAGGTGGGA-3′

PCR product was subjected to a restriction reaction using the BmgBI enzyme and run for analysis on DNA agarose gel.

### Protein Analysis

Since ApoE is mostly secreted from the cells, the medium of co-transfected cells was collected 3 days following co-transfection. An amount of 14 μl of medium from each sample was subjected to SDS gel electrophoresis and tested by western blot analysis using an goat anti-ApoE primary antibody at a concentration of 1:10,000 and a donkey anti-goat as a secondary antibody at a concentration of 1:5000 as previously described [14]. The immunofluorescence intensities were analyzed using Image Lab (BIORAD). Each group (ApoE3+/− treatment and ApoE4+/− treatment) had two biological repeats and each biological repeat had two technical repeats.

### Viral Constructs

LentiCRISPR V2 (Addgene #52961) plasmid was used as a backbone. In order to transform SpCas9 into the variant VRER SpCas9, (comprising amino acid sequence as denoted by SEQ ID NO 6, encoded by the nucleic acid sequence as denoted by SEQ ID NO. 5), the relevant mutations were introduced (by Biobasic) accordingly to the sequence of MSP1101 (D1135V/G1218R/R1335E/T1337R, Addegene #65773), the original plasmid of VRER SpCas9, as this plasmid was not suitable for viral construct production. The point mutations were introduced by fusion ligation. More specifically, the vector (LentiCRISPR V2 plasmid) was digested using EcoRV and BamHI firstly. The fragment containing the relevant point mutations was cloned into the vector through in fusion ligation. Thereafter, the sgRNA sequence was cloned into the vector as described previously [15]. More specifically, the vector (LentiCRISPR VRER SpCas9 plasmid) was digested by Esp3I enzyme and the linear plasmid was then purified by gel-electrophoresis. The gRNA fragment (double strand with sticky ends) with Esp3I's cohesive ends was cloned into the vector by annealing.

A lentiviral vector comprising a sequence encoding the gRNA of the invention and a nucleic acid sequence encoding the Cas9 variant with the following substitutions, specifically, D1135V/G1218R/R1335E/T1337R has been prepared. This vector is denoted by SEQ ID NO. 27.

The following sequences were annealed using PCR and then cloned:

(sense)
5′-CACCGGGCGCGGACATGGAGGACG-3′
as denoted by SEQ ID NO: 9
(anti-sense)
5′-AAACCGTCCTCCATGTCCGCGCCC-3′.
as denoted by SEQ ID NO: 10

For lentiviral production, the modified VRER LentiCRISPR V2 vector containing the sgRNA sequence for ApoE (having the nucleic acid sequence as denoted by SEQ ID NO: 27), the same vector without an sgRNA sequence, or a control plasmid were co-transfected with the packaging plasmids pLP1, pLP2, and pLP/VSVG into the 293T producer cell line using Lipofectamine 2000 (Invitrogen). The supernatant was collected 48 and 72 hours post transfection and was subsequently deposited using ultracentrifugation at 25,000 RPM for 2 hours. The virus-containing pellet was aspirated using HBSS, aliquoted, and stored at −80° C. until use. Lentiviral titer was determined using the Lenti-X p24 Rapid Titer Kit, following the manufacturer's procedure (Clontech Laboratories).

### Lentivirus Titer:

VRER SpCas9+ApoE gRNA: 1.26×10⁸ ifu
VRER SpCas9−no gRNA: 1×10⁸ ifu

### In-Vitro Lentiviral Transfection Experiment

Cells were grown to 80-90% confluency in 2 ml medium (6-well plates). An amount of 10 μl of the lentivirus vial were added to 50 ml fresh medium. The cultured medium of the cells was completely drawn and replaced with 0.5 ml of the fresh medium containing lentivirus. After 24 hours, the virus medium was drawn and replaced with fresh medium with lenti-particles. For the next 24 hours, the medium was replaced twice again. 24 hours later the cultured medium was collected for analysis of ApoE protein level and DNA was extracted from cells for further DNA analysis as described above.

### Mice Injections

Human ApoE target replacement (TR) mice were used comprising homozygous mice (E3/E3 or E4/E4) and heterozygous mice (E3/E4). For each genotype, the mice were divided into 3 groups: treatment, sham and naïve. Mice were anesthetized with a mixture of ketamine-xylazine and placed in a stereotactic apparatus. An amount of 2 μl of lentivirus preparation containing the CRISPR Cas system with or without the sgRNA were injected bilaterally into the CA3 region of the hippocampus using the following coordinates: ±2.3 mm medial/lateral, −2.1 mm anterior/posterior, and −2.2 mm dorsal/ventral from the bregma. The preparation was injected with a speed of 0.45 μl/min utilizing a Hamilton 10-μl syringe and a 26-gage needle. The mice were stitched and then returned to their cages. Injections were performed in 2 different sessions.

### Behavioral Experiments—Morris Water Maze

Cages were randomly encoded by a third-party agent and the researcher was blind for the genotypes and treatments of each cage.

The mice were tested 6 weeks post injections at the Morris water maze. The mice were placed in a 140 cm circular pool with water at 26^{c} rendered opaque with milk powder and with a 10 cm square platform submerged 1 cm below the surface of the water at a specific location. The mice were subjected to 4 trials per day for 4 days, were for each trial the mice were placed in a specific and different location along the perimeter of the pool. The order of the locations tested as well as the location of the platform were unchanged between days. At the first trial of day 1, the platform was introduced to each mouse for 20 seconds prior to the first trial. Each trial lasted 90 seconds unless the mouse reached the platform earlier. Once the mouse reached the platform or failed to do so in 90 seconds, the platform was left for 20 seconds and then taken out of the pool.

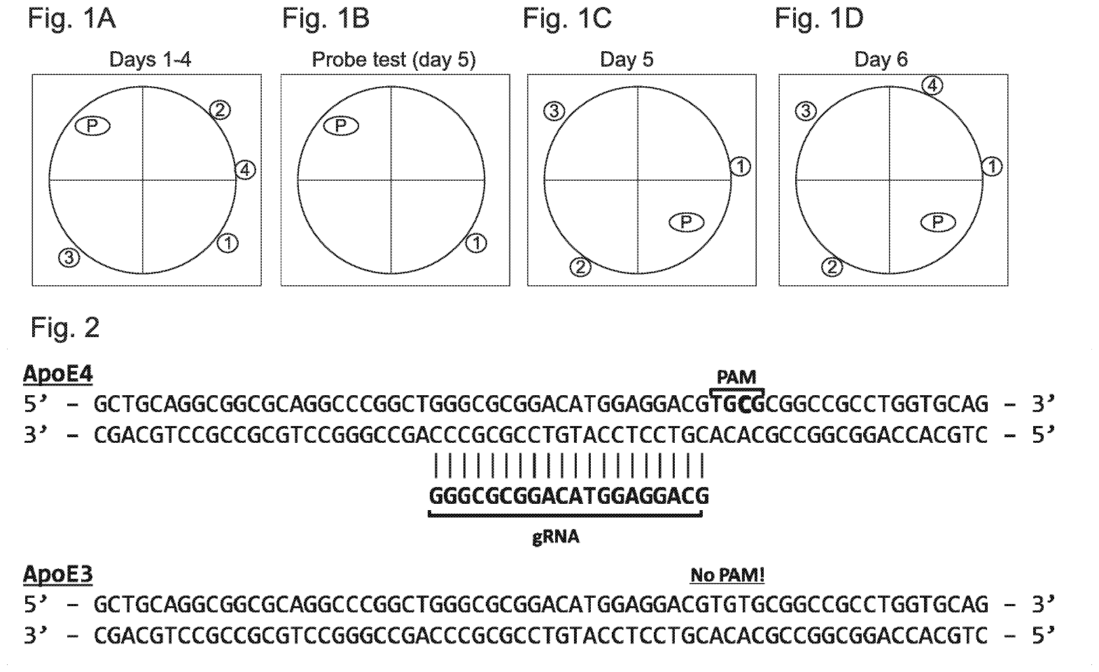

On the 5^{th} day, the platform was removed, and each mouse was given 90 seconds to seek for it (probe test). After the probe test, the platform was placed at the new location, opposite to its former location and for the next 3 trails on that day and more 4 trials at the day after, the mice went through the same procedure as the first 4 days. At the first trial after the probe test, each mouse was given 20 seconds on the platform. The locations of the platform and the start point of each trail are illustrated in FIG. 1.

The performance of the mice was monitored by measuring the time (seconds) they took to reach the platform. Measurements of the time to reach the platform were performed using the EthoVisionXT 11 program.

### Immunohistochemistry and Immunofluorescence Confocal Microscopy

One brain hemisphere of mice was fixed overnight with 4% paraformaldehyde in 0.1 M phosphate buffer, pH 7.4, and then placed in 30% sucrose for 48 h. Frozen coronal sections (30 um) were then cut on a sliding microtome, collected serially, placed in 200 μl of cryoprotectant (containing glycerin, ethylene glycol, and 0.1 M sodium-phosphate buffer, pH 7.4), and stored at −20° C. until use. Immunohistochemistry and innunofluorescence analysis was performed as previously described [14]. ApoE protein levels from the hippocampus were measured using an anti-ApoE antibody. DNA was extracted from the hippocampus for DNA analysis.

The pathological effects of ApoE4 were monitored using the following primary antibodies (Abs): mouse anti-neuN (1:500; Chemicon), guinea-pig anti-vesicular glutamatergic transporter 1 (VGluT1; 1:2000; Millipore), rabbit anti-apoER2 (CT kindly provided by J. Herz lab; 1:1000), rabbit anti-A42 (1:500; Chemicon, Temecula, Calif.), rabbit anti-202/205 phosphorylated tau (AT8; 1:200, Innogenetics). All the groups were stained together, and the results presented correspond to the mean±SEM of the percent area stained normalized relative to the naïve ApoE3 mice.

The immunostained sections were viewed using a Zeiss light microscope (Axioskop, Oberkochen, Germany) interfaced with a CCD video camera (Kodak Megaplus, Rochester, N.Y., USA). Pictures of stained brains were obtained at ×10 magnification. The staining was analyzed and quantified using the ImagePro plus system for image analysis (v. 5.1, Media Cybernetics, Silver Spring, Md., USA). The images were analyzed by marking the area of interest and setting a threshold for all sections subjected to the same staining. The stained area above the threshold relative to the total area was then determined for each section. All the groups were stained together and the results presented correspond to the mean±SEM of the percent area stained normalized relative to the young control apoE3 mice. Sections stained for immunofluorescence were visualized using a confocal scanning laser microscope (Zeiss, LSM 510). Images (1024×1024 pixels, 12 bit) were acquired by averaging eight scans. Control experiments revealed no staining in sections lacking the first Ab. The intensities of immunofluorescence staining were calculated utilizing the Image-Pro Plus system (version 5.1, Media Cybernetics) as previously described [14]. All images for each immunostaining were obtained under identical conditions, and their quantitative analyses were performed with no further handling. Moderate adjustments for contrast and brightness were performed similarly on all the presented images of the different mouse groups. The images were analyzed by setting a threshold for all sections having a specific labeling. The area of staining over the threshold relative to the total area of interest was determined and averaged for each mouse and each group, and was normalized to the ApoE3 naïve group.

### Example 1

### Design and Preparation of a Specific CRISPR System Targeting the APOE ε4 Allele

To specifically target and knockout the APOE ε4 allele, the CRISPR/Cas9 genome-editing platform was used. To design a specific cas9 system that destruct the APOE ε4 allele but cannot recognize the APOE ε3 allele that differ in only one base, a variant of Cas9 named spCas9 VRER was used [12]. This variant recognizes a unique sequence of PAM: NGCG which as illustrated by FIG. 2, appears only in the APOE ε4 locus, but not in the APOE ε3 locus, and thereby can be used to distinguish between the two isoforms. Thus, performing CRISPR/Cas9 genome-editing using the spCas9 VRER especially address the need of knocking-out the APOE ε4 allele without altering the APOE ε3 locus.

Relevant sgRNA (comprising the nucleic acid sequence as denoted by SEQ ID NO. 8) for targeting the APOE ε4 locus was produced by cloning into LRG plasmid, as described in experimental procedures.

Human ApoE3 or ApoE4 homozygous knock-in mouse astrocytic cells were then co-transfected with both spCas9 VRER and sgRNA encoding plasmids, and cultured as described in the Experimental procedures.

### Example 2

### Specificity of CRISPR Activity Against the APOE ε4 Locus Revealed by DNA Analysis

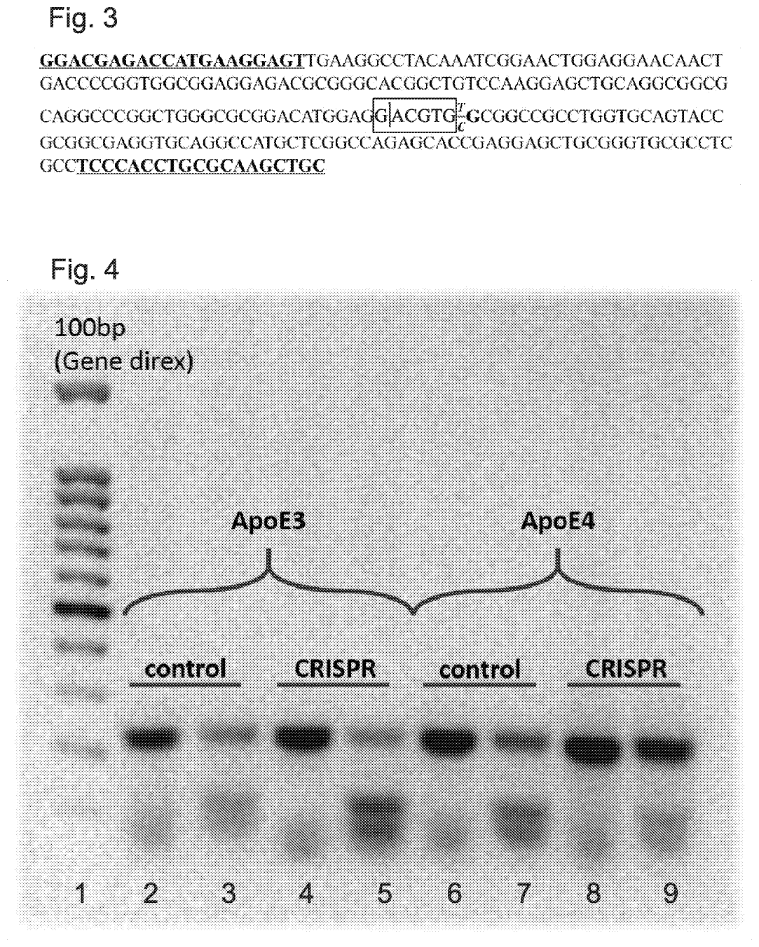

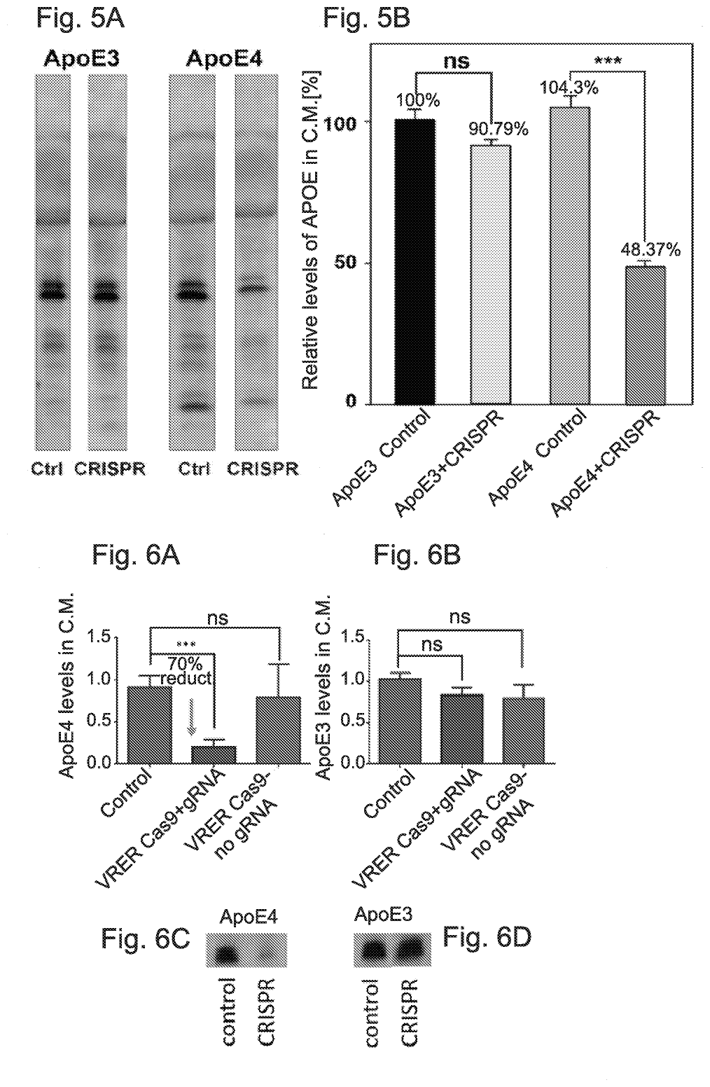

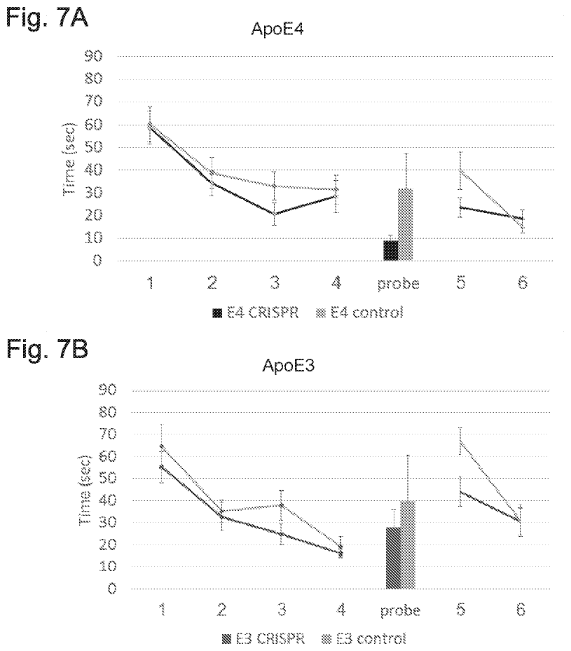

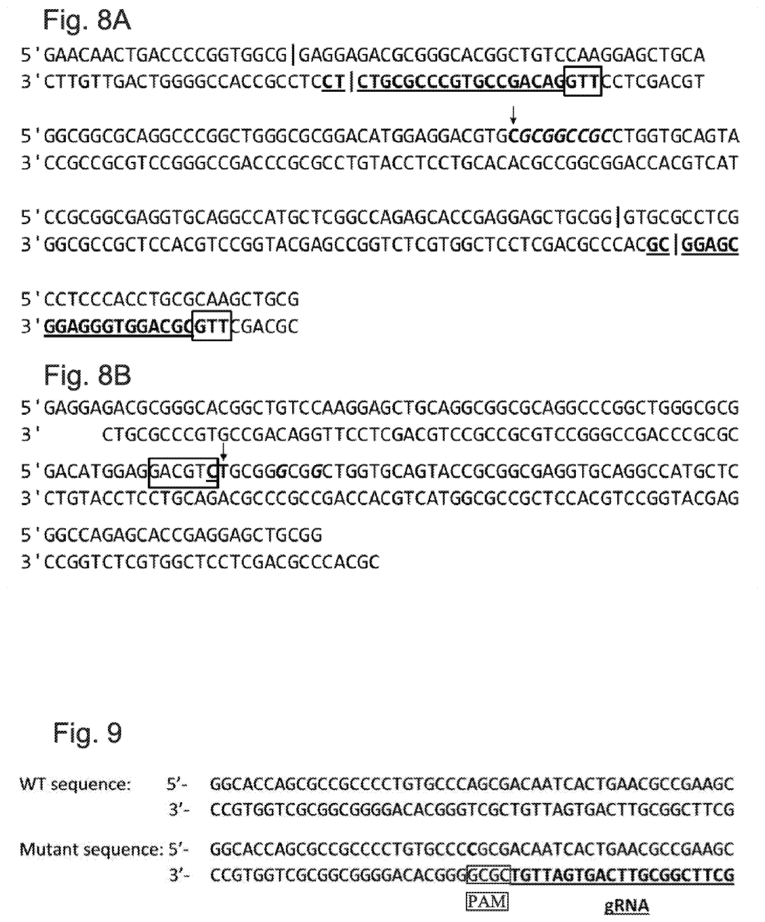

To verify the specific destruction of the APOE ε4 allele by the CRISPR system of the invention, a restriction enzyme that recognizes the GACGTG restriction site found exclusively in the APOE ε4 allele was used. DNA was extracted from above-described co-transfected cells and CRISPR activity was tested in the DNA samples. PCR was performed to amplify a 242 bp amplicon containing a part of ApoE gene. PCR product was then followed by a restriction reaction with the BmgBI enzyme which recognizes the restriction site CACGTC which should be present on the 242 bp PCR product described above. However following CRISPR specific targeting which causes insertion/deletion (indels) of about 3 nucleotides upstream to the PAM sequence, this restriction site is destroyed as schematically illustrated in FIG. 3. Thus, as a result of CRISPR activity, a full length amplicon is observed at the ApoE4 CRISPR—Cut sample (lane 9, FIG. 4) indicating that the restriction site has been specifically destructed, resulting in failure in restriction reaction. On the other hand, two major truncated bands are visible at the ApoE4 control—Cut group (lane 7, FIG. 4) indicating that when no CRISPR treatment is applied, the restriction reaction successfully occurs. The successful cleavage of the intact BmgBI restriction site within ApoE3 in the absence (control) or the presence of CRISPR, as presented by lanes 3 and 5, demonstrated the specificity of the CRISPR system of the invention to the ApoE4 allele. These results demonstrate the high specificity of the CRISPR system of the invention for knocking out the ApoE4 allele while allowing an intact APOE ε3 allele.

### Example 3

### The CRISPR System of the Invention Specifically Reduces Levels of APOE4 Protein

The inventors next compared the effect of the CRISPR system of the invention on APOE4 protein levels in comparison with the levels of APOE3 protein. The medium of co-transfected cells was collected three days after co-transfection for Western blot analysis. was As clearly demonstrated by FIG. 5, following CRISPR-Cas9 treatment, the amount of detected ApoE4 was reduced significantly (55.93%) whereas the detected ApoE3 levels were not affected by CRISPR-Cas9 activity. These result demonstrates the feasibility of the PAM specificity method of the invention in enabling specific knocking-out of only the ApoE4 protein without affecting the ApoE3 protein (in heterozygotes) even if these two isoforms differ by only one nucleotide and amino acid residue.

In-vitro experiments were further performed using a lentivirus vector as a delivery vehicle (the VRER SpCas9 containing sgRNA vector, as detailed in the experimental procedure comprising the nucleic acid sequence of SEQ ID NO. 27) in order to validate the performance of the CRISPR/Cas9 system. As illustrated in FIG. 6, even a higher rate of ApoE4 depletion was observed when using transfection with a lentivirus vector instead of a plasmid. ApoE4 protein levels in cells treated with the VRER SpCas9 containing sgRNA were decreased by 70% (FIG. 6A and FIG. 6C). In comparison, the levels of the ApoE3 were not affected by the CRISPR-Cas9 treatment (FIG. 6B and FIG. 6D).

### Example 4

### In Vivo Animal Model for Chronic Neurodegenerative Disorders

Encouraged by the results of the in vitro model described above, the inventor are testing also an in vivo model as follows: CRISPR containing lentiviral vector were injected intracerebrally to apoE4/apoE4, apoE3/apoE3 homozygous mice into the brain area which is the most affected by the apoE4 protein (e.g. the hippocampus). The efficacy of the treatment is assessed by checking if the APOE4 gene was destroyed thus leading to a substantial decrease of the level of the apoE4 protein in the brain without decreasing the levels of the apoE3 protein, similarly to the above-described experiments for the in vitro model (DNA and protein analysis). Specific improvements in the cognitive performance of the treated mice is also evaluated by performing the Morris water maze experiment, the Object recognition test as well as the Fear Conditioning test.

More specifically, the Morris water maze experiment was performed (see FIG. 7). The treated apoE4/apoE4 mice showed improved performance especially during the probe test at the 5^{th} day of the experiment (FIG. 7A). On the other hand, no significant change was observed for the apoE3/apoE3 mice treated mice during days 1-4 and the probe test. (FIG. 7B).

Reversal of the specific neuronal and synaptic impairments of the mice (e.g. increased levels of synaptophysin, the presynaptic transporters VgaT and VgluT and of the ApoE receptor apoER2) is also examined at the RNA-levels by performing RT-PCR analysis as well as at the protein levels by Western blot analysis and immunuhistochemistry staining of brain tissues as described in experimental procedures.

### Example 5

In Vivo Animal Model for Acute Insult Disorder To further examine the methods of the invention on acute neurodegenerative disorders that involved neuronal vascular and inflammatory aspects, brain injury originally described in the rat [Purushothuman et al PLOS (2013) 8(3) e5974] was adapted to the mouse utilizing a 25G needle which was inserted to the hippocampus at the coordinates: Ventral: −1.8 mm; lateral −1.5 mm and depth of 2 mm.

The mice are sacrificed at different time intervals up to 30 days following injury after which the brains are excised and the resulting brain pathology is assessed immunohistochemically. Parameters monitored include synaptic and vascular markers (e.g. synaptophysin and collagen IV) as well as brain inflammatory parameters such as GFAP and IBA1.

### Example 6

### Targeting of an Additional Pathological SNP in the ApoE Allele Using the CRISPR/Cas9 Genome-Editing Platform

The CRISPR/Cas9 genome-editing platform of the invention may be also applicable for other pathogenic SNPs in ApoE, specifically, pathogenic SNPs that form the specific PAM of the invention, specifically, the 5′-NGCG-3′ PAM. Thus, the inventors also use the method of the invention to specifically destruct the pathogenic APOE allele rs28931579 (at Position: 44909236 on chromosome 19), that was found to be associated with disorders similar to those associated with the ApoE4 allele. Here also, the spCas9 VRER a variant of Cas9 is used. This variant recognizes the PAM: NGCG which as illustrated by FIG. 9, appears only in the APOE rs28931579 allele, but not in the ApoE WT allele, and thereby can be used to distinguish between the two isoforms.

Relevant sgRNA (comprising the nucleic acid sequence as denoted by SEQ ID NO. 31) for targeting the APOE rs28931579 locus is produced by cloning into LRG plasmid or into a lentivirus vector (the VRER SpCas9 containing sgRNA vector) as delivery vehicles. Human APOE rs28931579 or WT ApoE homozygous knock-in mouse cells are then co-transfected with both spCas9 VRER and sgRNA encoding plasmids or with the VRER SpCas9 containing sgRNA vector and cultured. DNA analysis is performed to verify the specific destruction of the ApoE rs28931579 by the CRISPR system. In addition, the effect of the CRISPR system of the invention on APOE WT protein levels in comparison with the levels of APOE rs28931579 protein is examined. The medium of co-transfected/transfected cells is collected three days after co-transfection/transfection for Western blot analysis.

### Example 7

### The Use of SNP-Derived PAM as a Diagnostic Method Using the CRISPR/Cas System

The ability to distinguish between alleles and target only the ApoE4 allele or any other pathogenic allele of ApoE, out of the other alleles (ApoE2 and ApoE3) as provided by the invention, enables not only to cause knockout of the ApoE4 protein, but also to detect it using a mutated Cas protein (dead-Cas) that recognizes the specific PAM of the invention, and is directly or indirectly contacted with a detectable moiety, for example, and a green-fluorescent protein (GFP) that may be either fused to it or connected to the gRNA used.

More specifically, dead-Cas9 (dCas) is a variation of the Cas9 protein, in which point mutations (D10A and H840A) lead to loss of function of the two active nuclease sites of the Cas9 (RuvC and HNH domains). Thus, the dCas is no longer capable of DNA cleavage; however, it can still bind RNA and form the Cas9:gRNA complex and therefore bind the target DNA.

Thus, in some embodiments, dCas suitable in the present invention may be a cas9 variant comprising at least one of Valine at position 1135 (1135V), Arginine at position 1218 (1218R), Glutamine at position 1335 (1335G) and Arginine at position 1337 (1337R). As indicated above, the detectable moiety may be either fused directly to the Cas protein used, or alternatively, indirectly bound via aptamers such as MS2, PP7 and other RNA binding motifs commonly used to attach detectable moieties such as the GFP to the dCas, for example, via the gRNA. It should be appreciated that this version enables the use of multiple detectable moieties (e.g., in different colors), and thus, may provide detection of more than one target sequence, for example, more than one SNP in the ApoE gene. In brief, the MS2 RNA binding motif is added as an RNA sequence to the gRNA 3′ end. MS2-binding protein (MBP) fused to a detectable moiety, for example, fluorophore (e.g. GFP, RFP, BFP and the like) binds the MS2 domain. All together a complex of dCas9:gRNA:GFP is obtained. When the complex attaches the DNA target, the detectable moiety GFP can be visualized and indicates the appearance of a specific sequence, guided by the gRNA or in this case, by both the gRNA and PAM sequences.

The VRER SpCas9 is thus altered to become a “dead VRER SpCas9” and fused to GFP as described in order to perform a method of detecting the ApoE4 allele in genetic tests.

This application is efficient and profitable when using the visualization possibility of CRISPR as a genetic screening method. One can test hundreds or even more gRNA sequences fused to different fluorophores to detect genetic variations from a DNA test.

### Example 8

### ApoE4 to ApoE3 Correction Using CRISPR Cpf-1

In yet a further alternative approach for editing ApoE4 in the brain, a Cpf-1 based CRISPR system is designed, targeted with two gRNAs sequences upstream and downstream to the ApoE4 SNP, as also illustrated by FIG. 8A. By targeting these sequences, Cpf-1 creates double stranded staggered breaks in the genomic DNA, allowing a “Donor”, a dsDNA sequence, to fill the gap by its sticky ends (FIG. 8B). The donor sequence includes the corrected nucleotide (C to T). Additionally, in order to enable further analysis, a restriction enzyme recognition sequence (AatII, GACGTG) is further added (by replacing the nucleotide sequence without altering the resultant amino acid sequence), and whereas another restriction enzyme' recognition sequence (NotI, GCGGCCGC) is deleted.

1. A method for targeted elimination of at least one Apolipoprotein E (ApoE) pathogenic protein in a cell, the method comprising the step of contacting said cell with an effective amount of:
(a) at least one polypeptide comprising at least one clustered regulatory interspaced short palindromic repeat (CRISPR) associated (cas) protein, or any nucleic acid encoding said polypeptide, wherein said cas protein specifically recognizes the 5′-NGCG-3′ proto-spacer adjacent motif (PAM); and
(b) at least one nucleic acid sequence comprising at least one guide RNA (gRNA) that targets a protospacer located upstream to said PAM within at least ApoE pathogenic allele encoding said at least one pathogenic ApoE protein, or any nucleic acid sequence encoding said gRNA; or with a kit, composition or vehicle comprising (a) and (b).

2. The method according to claim 1, for targeted elimination of the Apolipoprotein E 4 (ApoE4) protein in a cell, the method comprising the step of contacting said cell with an effective amount of:
(a) at least one polypeptide comprising at least one Cas protein, or any nucleic acid encoding said polypeptide, wherein said cas protein specifically recognizes the 5′-NGCG-3′ PAM; and
(b) at least one nucleic acid sequence comprising at least one gRNA that targets a protospacer located upstream to said PAM within the ApoEε4 allele, or any nucleic acid sequence encoding said gRNA; or with a kit, vehicle or composition comprising (a) and (b).

3. The method according to claim 1, wherein said cas protein is a member of at least one of CRISPR-associated system of Class 1 and Class 2, wherein said cas protein is a member of a CRISPR-associated system type II of class 2, optionally, said Cas protein is CRISPR associated protein 9 (cas9) variant that specifically recognizes the 5′-NGCG-3′ PAM, or any derivative or fusion protein thereof.

4-5. (canceled)

6. The method according to claim 3, wherein said Cas9 variant comprises at least one of Valine at position 1135 (1135V), Arginine at position 1218 (1218R), Glutamine at position 1335 (1335G) and Arginine at position 1337 (1337R).

7. The method according to claim 6, wherein said Cas9 variant is the Streptococcus pyogenes Cas9 (SpCas9) VRER variant or any derivative or fusion protein thereof, optionally, said SpCas9 VRER variant comprises the amino acid sequence as denoted by SEQ ID NO: 6, or any derivative or fusion protein thereof.

8. (canceled)

9. The method according to claim 1, wherein said gRNA targets a protospacer comprising the nucleic acid sequence as denoted by SEQ ID NO: 18 of the ApoEε4 allele, or any fragments thereof, optionally, said gRNA comprises the nucleic acid sequence as denoted by SEQ ID NO: 8.

10-11. (canceled)

12. The method according to claim 1, for treating, preventing, ameliorating, inhibiting or delaying the onset of a pathologic disorder associated with at least one pathogenic form of the Apo E protein in a mammalian subject, said method comprises the step of administering a therapeutically effective amount of:
(a) at least one polypeptide comprising at least one Cas protein, or any nucleic acid encoding said polypeptide, wherein said cas protein specifically recognizes the 5′-NGCG-3′ PAM; and
(b) at least one nucleic acid sequence comprising at least one gRNA that targets a protospacer located upstream to said PAM within the pathogenic form of the ApoE allele encoding said at least one pathogenic ApoE protein, or any nucleic acid sequence encoding said gRNA; or a kit or composition comprising (a) and (b).

13. The method according to claim 12, for treating, preventing, ameliorating, inhibiting or delaying the onset of an ApoE4 associated pathologic condition or disease in a mammalian subject, wherein said mammalian subject carries at least one ApoEε4 allele, and wherein said method comprises the step of administering to said subject a therapeutically effective amount of:
(a) at least one polypeptide comprising at least one Cas protein, or any nucleic acid encoding said polypeptide, wherein said cas protein specifically recognizes the 5′-NGCG-3′ PAM; and
(b) at least one nucleic acid sequence comprising at least one gRNA that targets a protospacer located upstream to said PAM within the ApoEε4 allele, or any nucleic acid sequence encoding said gRNA; or a kit or composition comprising (a) and (b).

14. The method according to claim 13, wherein said ApoE4 associated condition or disease is:
(a) at least one of: an acute or chronic vascular, inflammatory and neurodegenerative pathology or condition or any combination thereof; or
(b) poor outcome following traumatic brain injury (TBI).

15. The method according to claim 14, wherein at least one of:
(a) said neurodegenerative disorder is Alzheimer's disease;
(b) said vascular pathology or condition is at least one of cerebrovascular condition or disease and cardiovascular condition or disease, optionally, said cerebrovascular condition or disease comprises vascular, cognitive impairment disorders and conditions:
(c) said at least one of neurodegenerative, inflammatory, or vascular impairments comprises Mild cognitive impairment (MCI), Age-related cognitive decline and Dementia with Lewy bodies (DLB), and tauopathies; and
(d) said vascular, inflammatory, or neurodegenerative condition comprises at least one of accelerated age-related decrease in cortical thickness and hippocampal volume, Cerebral angiopathy, Impaired synaptic plasticity, synaptic loss, hippocampal atrophy, loss of dendritic spines, brain inflammation, neurovascular dysfunction, BBB-breakdown, leakage of blood derived toxic proteins into the brain and reduction in the length of small vessels, Frontotemporal dementia, Neuro-inflammation, and impairment of neurogenesis.

16-21. (canceled)

22. The method according to claim 12, wherein said Cas protein is a member of at least one of CRISPR-associated system of class 2 and class 1, wherein said cas protein is a member of a CRISPR-associated system type II of class 2, and optionally, wherein said Cas protein is Cas9 variant that specifically recognizes the 5′-NGCG-3′ PAM, or any derivative or fusion protein thereof.

23-24. (canceled)

25. The method according to claim 22, wherein said Cas9 variant comprises at least one of Valine at position 1135 (1135V), Arginine at position 1218 (1218R), Glutamine at position 1335 (1335G) and Arginine at position 1337 (1337R).

26. The method according to claim 25, wherein said Cas9 variant is the SpCas9 VRER variant or any derivative or fusion protein thereof, optionally, said SpCas9 VRER variant comprises the amino acid sequence as denoted by SEQ ID NO: 6 or any derivative or fusion protein thereof.

27. (canceled)

28. The method according to claim 12, wherein said gRNA targets a protospacer comprising the nucleic acid sequence as denoted by SEQ ID NO: 18 of the ApoEε4 allele, or any fragments thereof, optionally, said gRNA comprises the nucleic acid sequence as denoted by SEQ ID NO: 8.

29. (canceled)

30. A pharmaceutical composition comprising a therapeutic effective amount of:
(a) at least one polypeptide comprising at least one Cas protein, or any nucleic acid encoding said polypeptide, wherein said Cas protein specifically recognizes the 5′-NGCG-3′ PAM; and
(b) at least one nucleic acid sequence comprising at least one gRNA that targets a protospacer located upstream to said PAM within a pathogenic allele of ApoE or any nucleic acid sequence encoding said gRNA; or
(c) a kit comprising (a) and (b);
said composition optionally further comprises at least one of pharmaceutically acceptable carrier/s, diluent/s and/or excipient/s.

31. The pharmaceutical composition according to claim 30, comprising a therapeutic effective amount of:
(a) at least one polypeptide comprising at least one cas protein, or any nucleic acid encoding said polypeptide, wherein said cas protein specifically recognizes the 5′-NGCG-3′ PAM; and
(b) at least one nucleic acid sequence comprising at least one gRNA that targets a protospacer located upstream to said PAM within the ApoEε4 allele, or any nucleic acid sequence encoding said gRNA; or
(c) a kit comprising (a) and (b);
said composition optionally further comprises at least one of pharmaceutically acceptable carrier/s, diluent/s and/or excipient/s.

32-33. (canceled)

34. The composition according to claim 31, wherein said Cas protein is CRISPR associated Cas9 variant that specifically recognizes the 5′-NGCG-3′ PAM, or any derivative or fusion protein thereof, wherein said Cas9 variant comprises at least one of Valine at position 1135 (1135V), Arginine at position 1218 (1218R), Glutamine at position 1335 (1335G) and Arginine at position 1337 (1337R).

35. (canceled)

36. The composition according to claim 34, wherein said Cas9 variant is the SpCas9 VRER variant or any derivative or fusion protein thereof, optionally, said SpCas9 VRER variant comprises the amino acid sequence as denoted by SEQ ID NO. 6, or any derivative or fusion protein thereof.

37. (canceled)

38. The composition according to claim 31, wherein said gRNA targets a protospacer comprising the nucleic acid sequence as denoted by SEQ ID NO: 18 of the ApoEε4 allele, or any fragments thereof, optionally, said gRNA comprises the nucleic acid sequence as denoted by SEQ ID NO: 8.

39-43. (canceled)

44. A diagnostic kit comprising:
a. at least one polypeptide comprising at least one dCas, or any nucleic acid encoding said polypeptide, wherein said dCas protein specifically recognizes the 5′-NGCG-3′ PAM; and
b. at least one gRNA that targets a protospacer located upstream to said PAM within the ApoE pathogenic allele, or any nucleic acid sequence encoding said gRNA;
wherein at least one of said dCas of (a) and said gRNA of (b) is directly or indirectly attached to at least one detectable moiety, wherein said ApoE pathogenic allele is ApoEε4 allele.

45. (canceled)
